(19) United States
(12) Patent Application Publication
(10) Pub. No.: US 2003/0135174 A1
(43) Pub. Date: Jul. 17, 2003

(54) Products with color masking properties

(76) Inventors: Herman P. Benecke, Columbus, OH (US); William A. Ivancic, Columbus, OH (US); Virginia M. Northrup, Columbus, OH (US); David Tipple, Columbus, OH (US); J. Michael Spangler, Reynoldsburg, OH (US); Jean E. Schelhorn, Granville Township, OH (US); James M. Sonnett, Upper Arlington, OH (US); Elizabeth Drotleff, Worthington, OH (US); C. Michael Gegenheimer, Upper Arlington, OH (US); Jeffrey Cafmeyer, Columbus, OH (US)

Correspondence Address: MACMILLAN, SOBANSKI & TODD, LLC, ONE MARITIME PLAZA - FOURTH FLOOR, 720 WATER STREET, TOLEDO, OH, 43604, US

(21) Appl. No.: 10/211,989

(22) Filed: Aug. 2, 2002

Related U.S. Application Data
(60) Provisional application No. 60/309,908, filed on Aug. 6, 2001.

(51) Int. Cl. A61L 2/00; B05D 3/00; B05D 1/32; B05D 1/36; B05D 5/00; A61F 13/15; A61F 13/20

(52) U.S. Cl. 604/367; 604/382; 604/385.01; 427/2.31; 427/259

(57) ABSTRACT

An adsorbent fabric includes a color masking layer including fluid impermeable areas disposed on a fluid permeable support fabric in spaced relationship.

RELATED APPLICATIONS

This application claims priority to U.S. Provisional Patent Application Serial No. 60/309,908, filed Aug. 3, 2001.

### TECHNICAL FIELD

This invention relates generally to products for absorbing to bodily fluids such as feminine sanitary pads, tampons, wound dressings, bandages, and the like. More particularly, the present invention relates to products for absorbing body fluids that are adapted to effectively reduce the visual perception of blood or other bodily fluids.

### BACKGROUND OF THE INVENTION

Various personal and disposable absorbent articles are well known and are in wide use. Examples of such widely used absorbent articles include feminine sanitary pads, adult incontinence products, diapers, tampons, and bandages. For example, absorbent products such as feminine sanitary pads and bandages configured for the absorption of bodily fluids are well known and in wide use in most developed countries.

Most sanitary pads in use today comprise an absorbent element or absorptive core disposed between an upper liquid permeable containment layer, more simply referred hereafter as a containment layer, and a lower liquid impervious protective barrier. The containment layer can be comprised of a woven, nonwoven, or webbed fabric or layer and can be comprised of either hydrophobic or hydrophilic materials. One role of the containment layer is to encase the loose or bound absorptive fibers of the absorptive core and to provide structural support to the sanitary pad. When the containment layer is composed of hydrophobic materials, it also functions to transfer the released fluid from the wearer to the absorptive core while providing a relatively dry feeling to the wearer.

Optionally, a woven, non-woven, or webbed fabric or layer comprised of hydrophobic material may be positioned above the containment layer to provide additional comfort to the wearer due its non-retention of fluid, thus contributing to a dry feeling. Such fabric or layer will be referred to herein as a “topsheet”. Optionally, a spreading layer may be positioned between the containment layer and the absorptive core to help distribute catamenial fluid and other vaginal discharges more evenly to the absorptive core. The absorptive core is adapted to receive and contain catamenial fluid and other vaginal discharges. The protective barrier, or backsheet, is intended to prevent catamenial fluid and other vaginal discharges from passing through the absorbent core and from soiling the wearer's clothing.

Other commercially available absorbent articles, such as diapers, incontinence products, bandages, and perspiration pads, work in much the same manner as the above-described typical feminine sanitary pad. A liquid permeable layer facing the user allows for blood, urine and other bodily fluids to pass therethrough and be stored in an absorptive core. For example, a typical bandage incorporates a liquid impervious backsheet that protects the wearer's clothing from fluid seepage.

Various configurations and designs of liquid permeable containment layers are in use and known in the prior art. Typically, the body-facing containment layer has been made from a soft, non-woven fabric that allows fluid to migrate into the absorbent core while, when prepared from hydrophobic materials, separating the body from the discharge to keep the skin clean and dry. A key disadvantage associated with most prior art containment layer materials is that often some fluid is retained in the structure of this layer. This results not only in a wet surface being disposed against a wearer, but also in the red color of menstrual fluid being visually evident in this layer by the user after removal of the pad. Furthermore, the design of most prior art feminine hygiene pads (and bandages) allows the user of spent products to also see through the open structure of the topsheet and containment layer and observe the red color of blood or menstrual fluid within the lower spreading layer or within the absorptive core. This observation of red color of menstrual fluid within used feminine pads has been identified as being unsightly, unaesthetic, and undesirable.

The prior art describes many visual blocking approaches to overcome the disadvantage associated with user observation of red menstrual fluid in spent feminine hygiene pads. U.S. Pat. No. 5,620,741 employs small hollow cyclic hydrophobic protrusions on a hydrophilic containment layer to provide line-of-sight visual blocking. U.S. Pat. No. 5,261,899 describes a containment layer comprised of three layers, one of which contains opacifying fillers for visual obscuration. U.S. Pat. No. 5,158,819 describes use of hydrophobic microbubbled surface aberrations that provide line-of-sight visual blocking.

U.S. Pat. No. 5,078,710 describes use of a containment layer composed of a hydrophobic opaque film stamped with apertured recesses having steep sides to provide line-of-sight visual blocking. U.S. Pat. No. 5,693,037 describes the use of two hydrophobic layers containing capillaries allowing liquid flow so that the non-alignment of capillaries results in line-of-sight visual blocking. EP Pat. Appl. Nos. EP 1 174 101 A1 and EP 1108406A2 are directed towards absorbent articles, sanitary pads in particular, that are dark colored so as to match and not to be easily apparent when worn with similarly dark colored undergarments and clothing.

Nonetheless, the above-described approaches have not fully resolved the problem of effectively providing visual masking of absorbed fluids in a used sanitary pad from a viewer. Because of the above-described and other shortcomings of prior art absorbent articles, there has been a long felt need for absorbent articles such as sanitary pads that are both effective for absorbing and retaining fluids and that are also advantageously adapted to effectively reduce the visual perception of the presence of such fluid from an observer or user.

### BRIEF DESCRIPTION OF THE DRAWINGS

The invention will be described below with reference to the accompanying drawings, in which:

FIG. 1 is a top plan view of a typical preferred prior art sanitary pad adapted for use with the present invention;

FIG. 2 is a lateral cross sectional view of a first embodiment of the sanitary pad incorporating a color masking layer of the present invention;

FIG. 3 is a lateral cross sectional view of a second embodiment of the sanitary pad incorporating a color masking layer of the present invention;

FIG. 4 is a lateral cross sectional view of a third embodiment of the sanitary pad incorporating a color masking layer of the present invention;

FIG. 5 is a lateral cross sectional view of a fourth embodiment of the sanitary pad incorporating a color masking layer of the present invention;

FIG. 6 is a lateral cross sectional view of a fifth embodiment of the sanitary pad incorporating a color masking layer of the present invention;

FIGS. 7A-C are sequential lateral diagrammatic cross sectional views showing bodily fluid coming into contact and being contained within a typical white prior art sanitary pad;

FIG. 8 is a top plan diagrammatic view of the sanitary pad with contained fluid shown in FIG. 7C;

FIGS. 9A-C are sequential lateral diagrammatic cross sectional views showing bodily fluid coming into contact and being contained within a colored sanitary pad;

FIG. 10 is a top plan diagrammatic view of the sanitary pad with contained fluid shown in FIG. 9C;

FIGS. 11A-C are lateral cross sectional views of various alternative embodiments of the sanitary pad further incorporating a color masking layer of hydrophobic surfaces disposed on a layer of support fabric;

FIG. 11D is a lateral cross sectional view of an alternative embodiment of the sanitary pad incorporating a layer of color masking hydrophobic surfaces directly on a spreading layer;

FIG. 12 is a bar graph showing ΔE values for a number of hydrophilic surfaces;

FIG. 13 is a graph showing the relationship between L values and AE values for a number of hydrophilic surfaces;

FIGS. 14A-C are sequential lateral diagrammatic cross sectional views showing bodily fluid coming into contact and being contained within another embodiment of a sanitary pad of the present invention which incorporates a layer of color masking hydrophobic surfaces;

FIG. 15 is a top plan diagrammatic view of the sanitary pad with contained fluid with a layer of hydrophobic color masking surfaces fluid shown in FIG. 14C;

FIGS. 16 is a top diagrammatic view showing one preferred spacing arrangement and size of the color masking hydrophobic surface layer of one embodiment of the present invention;

FIG. 17A is a graph depicting the relationship between ΔE and L_{sytem}−L_{cloth}/L_{system};

FIG. 17B is a bar graph that depicts observed ΔE values for samples with (actual) and without (ideal) residual fluid on hydrophobic surfaces;

FIG. 18 is a bar graph showing observed ΔE values for different color masking layer samples all incorporating 0.3 mm diameter hydrophobic surfaces;

FIG. 19 is a bar graph showing observed ΔE values for different color masking layer samples all incorporating 0.5 mm diameter hydrophobic surfaces;

FIG. 20 is a bar graph showing observed ΔE values for different color masking layer samples all incorporating 1.0 mm diameter hydrophobic surfaces; and

FIG. 21 is a bar graph showing observed ΔE values for different color masking layer samples all incorporating 2.0 mm diameter hydrophobic surfaces;

FIG. 22 is a roll of fabric treated or made according to the invention;

FIG. 23 is shirt treated or made according to the invention;

FIG. 24 is a necktie treated or made according to the invention;

FIG. 25 is a diaper treated or made according to the invention;

FIG. 26 is a surgical face mask treated or made according to the invention;

FIG. 27 is an upholstered chair treated or made according to the invention;

FIG. 28 is a garage floor mat treated or made according to the invention; and

FIG. 29 is a food tray with an absorbent mat treated or made according to the invention.

### DETAILED DESCRIPTION OF THE PREFERRED EMBODIMENT

Preferred operative embodiments espousing the principle objects of this invention will now be described. The color masking device of the present invention has application to essentially any commercially available absorbent article such as sanitary pads, incontinence products, perspiration pads, bandages, and diapers. For illustrative purposes, the present invention is primarily described in the context for use with a feminine sanitary pad. However, it should be appreciated that any of the aforementioned products will benefit from and can incorporate the teachings of the present invention. Accordingly, the examples shown and described herein should be considered illustrative of the invention and not as restrictive.

According to an important aspect of the present invention, a feminine sanitary pad is provided with a layer of color masking material. The layer of color masking material, as will be described in detail below, is adapted to reduce the visual perception of the red color of menstrual fluid while not interfering with the functionality of the sanitary pad. As described previously, one general approach to obscure the presence of menstrual fluid described in the prior art is to provide thin, hydophobic opaque films on top of a sanitary pad. Products having such films have not been widely accepted because of slow fluid absorption rates and the relatively uncomfortable feeling of the hydophobic film. The color masking layer of the present invention in contrast is adapted to effectively mask the red color of menstrual fluid while not decreasing user comfort or decreasing fluid absorption properties.

The absorbent disposable articles contemplated for use with the present invention generally comprise a liquid permeable containment layer which further comprises the wearer or user facing surface, a backsheet which provides the outer or possibly the garment facing surface, and an absorbent core disposed there between. Optionally, a liquid permeable and hydrophobic woven, non-woven, or webbed fabric topsheet may be disposed on top of the containment layer. Optionally, a spreading layer may be positioned between the containment layer and the absorptive core to help distribute menstrual fluid more evenly to the absorptive core.

A typical liquid-impermeable backsheet is designed to prevent bodily fluids contained in the absorbent structure from wetting articles that contact the absorbent products such as a user's clothing. In effect, a typical backsheet acts as a barrier to fluid flow. Accordingly, the backsheet extends across the entire surface of the absorbent structure and may also form part of any side flaps or side wrapping elements.

Various materials known in the art are suitable for use as backsheet material including woven or non-woven hydrophobic materials such as polymeric films (including polyethylene or polypropylene thermoplastic films) or composite material such as film-coated non-woven material. In a typical sanitary pad backsheet, the polyethylene backsheet film has a thickness of less than 3.0 mils. A backsheet may be embossed finished to provide a more cloth-like and appealing appearance.

Attached to and disposed on the backsheet of a typical sanitary pad is an absorbent core. The color masking layer of the present invention may be used in combination with a sanitary pad incorporating essentially any of the absorbent core systems known in the art. The term absorbent core herein refers to any material or multiple layers of material having the primary function to absorb, distribute, and store menstrual fluid.

A spreading layer may be positioned above the absorptive core and underlie the containment layer. A typical fluid spreading layer is adapted to transfer menstrual fluid from physiologically localized areas on the containment layer to nearly the entire length and width of the absorptive core so as to maximize the fluid absorption efficiency and capacity of the underlining fluid storage layers or absorbent core.

The absorptive core or fluid storage layers generally comprises absorbent material including cellullosics such as cotton and may contain absorbent gel materials such as hydrogels and hydrocolloidal materials. Preferable gel materials for use in the absorbent core are adapted to absorb large quantities of body fluids and are capable of retaining such fluids under pressure.

Absorbent gel materials can be dispersed homogeneously or non-homogeneously in suitable gel carrier materials. Gel materials for use with a sanitary pad will most often comprise substantially water swellable and insoluble, crossed-linked, polymeric gel material. Carriers for holding the gel materials may be natural, modified or synthetic fibers, or non-modified cellulose fibers. Preferably, the carrier materials that hold the gel materials are hydrophilic or are treated to be hydrophilic so as to draw the fluid more effectively into the absorbent core.

As discussed previously, a liquid permeable containment layer is disposed on top of the absorbent core. The containment layer is preferably pliable and nonirritating to a wearer's skin. This layer may also be comprised of materials exhibiting elastic characteristics allowing it to be stretched in different directions. This containment layer must also be fluid permeable, thus allowing bodily fluids to readily penetrate through the containment layer's thickness. The containment layer may be comprised of a wide range of materials such as woven, non-woven, or webbed hydrophobic or hydrophilic materials, polymeric materials such as apertured formed thermoplastic films, apertured plastic films, and hydroformed thermoplastic films. Suitable woven and non-woven materials may further be comprised of natural fibers, synthetic fibers, or from a combination of natural and synthetic fibers. Examples of preferred synthetic fibers include relatively hydrophilic polymeric fibers made from various polyesters, and hydrophobic fibers or webs prepared from polyolefins such as polypropylene or polyethylene fibers. If the containment layer is prepared from hydrophobic fibers or webbed structures, it provides increased wearer comfort due to decreased retention of fluid in this layer. If the containment layer is prepared from hydrophilic fibers or webbed structures, liquid transfer through the containment layer to the absorptive core may proceed at a faster rate. However, use of such hydrophilic materials could result in a wet feeling to the user due to the wetability of such materials. A typical containment layer extends across the whole of the absorbent structure.

Additionally, a topsheet may be attached above the top of the containment layer described above that makes direct contact with the pad wearer. The topsheet is composed of a hydrophobic webbed structure or woven or non-woven fibers prepared from hydrophobic materials. Typical webbed topsheets are composed of polyolefins such as polyethylene or polypropylene. Menstrual fluid contacting the topsheet structure will not be retained and will be transferred to the underlying containment layer, thus providing a relatively dry feel to the wearer. Topsheets will be advantageously deployed when the containment layer is comprised of hydrophilic woven or non-woven fabrics or hydrophilic webbed structures due to the enhanced wetability of these hydrophilic materials. A typical topsheet extends across the whole of the absorbent structure.

In a typical sanitary pad, the topsheet, containment layer, spreading layer, absorbent core, and backsheet components are joined together to form a usable article. Various elements and layers are joined together in any one of several acceptable methods known in the art including providing continuous layers of adhesive, pattered layers of adhesive, heat bonding, or mechanical bonding. The containment layer may also be joined to the backsheet about the periphery of the absorbent article. Additionally, the garment-facing surface of the backsheet of the sanitary pad may be provided with a layer of adhesive for adhesively joining the sanitary pad to an undergarment.

Now that the typical construction of a sanitary pad usable with the color masking layer of the present invention has been described, it is instructive to consider how the color masking layer operates. The color masking layer of the present invention, in its simplest form, uses visual blocking mechanisms to reduce the visual perception of an observer and to prevent a user from seeing colored stains that result from residual blood or menstrual fluid contained in a sanitary pad.

Standards and Measurements to Define the Observations of the Eye

Visual blocking is generally defined as those modifications made to sanitary pads that prevent the eye from seeing menstrual fluid deposited on and being retained in the pad. Descriptions of visual blocking in the prior art typically employ pad construction devices that either block the line of sight between the observer and the red or stained components of the sanitary pad or employ and use opaque materials to prevent seeing the red color of red stained components within the pad. However, one embodiment of the color masking approach of the present invention seeks to control perceived images in a completely different manner through the use of variable sized components having different colors and reflectance values. The principles and performance of the principle invention will become evident by consideration of the figures and explanations that follow.

The perceived color of an object depends on that material's characteristic absorption and reflectance of different frequencies of electromagnetic radiation in the visible region of the spectrum. For example, if a material's surface is totally reflective (i.e., it reflects all radiation in the visible region), that material will appear as white. If the material reflects light rays in the red region of the visible spectrum while absorbing all other light frequencies, that material will be perceived as red.

As shown in Table 1 below, observations of effects of adding simulated menstrual fluid to the colored fabrics and colored hydrophobic surfaces (the purpose of which will be described in detail below), are described in terms of an observer by the following terms: Not observable, barely observable, low contrast observable, easily observable, sharply contrasting. These observations correspond to quantifiable ΔE values.

Color measurements provided in this discussion use the CIE LA*B (LAB) color system. The (LAB) color system, that was first described by Hunter in 1942 and has become a standard method of measuring color, is controlled by the International Commission on Illumination (CIE) and supported by the US NIST which is a member organization. The LAB system quantifies visual changes that are calibrated to visual changes perceived by a standard observer. The LAB system, using illumination with a D65 standard light, was used by making spectroscopic measurements of the differences in reflectance between sample color systems and the same sample color systems that had been challenged with simulated menstrual fluid.

The LAB system correlates to the sensitivity of the eye towards brightness or luminance and to color changes. The L value reflects the eye's sensitivity to brightness as one observes colors ranging from dark colors to bright color and from gray to white. L is defined as 0 for a surface that is non-reflecting black and 100 for a surface that is non-fluorescing white. During fluorescence, where light is transferred from a wavelength that is less sensitively seen by the eye to one in which light is more sensitivity observed, L can be measured as greater than 100, where the object is brighter than the source. The variable a* represents the ratio of the reflectance in the green region to the reflectance in the red region and the variable b* represents the sensitivity to the ratio of the reflectances in the blue region to the yellow regions of the electromagnetic spectrum. The values for the variables L, a*, and b* are calculated from reflectance spectra.

Black is lack of color. In the extreme it is L=0 and a*=0 and b*=0. Practically black objects have some surface reflectance. As defined herein, black includes the effects of surface reflectance, and black is defined as L<25 with a*and b* near zero (<2). Examples discussed herein include black materials where L=17, which materials were nominally glossy, and materials where L=23 for a highly glossy source. Very flat blacks will have lower L values.

Color, typically includes every value of color space which includes black. For purposes of this application, the definition of color will exclude black, but includes whites (L>85) and grays (25<L<85 with a* and b* small (less than <1)), as grays have low chromaticity. The concept of color includes chromaticity >0, which is the imbalance in the relative reflectance of individual spectral regions as compared to a white standard. Examples of color are violet, blue, light blue, green, yellow, orange red, and combinations of wavelengths that include brown, maroon, and combination colors. Saturated colors have high values of a* or b*, such as the colors of the rainbow. Unsaturated colors have lower chromaticity, characterized by lower values of a* and b*. Some examples of unsaturated colors are light blue, light green, light yellow and pink (typically having L>50). Unsaturated colors with L<50 can also have lower chromaticity, lower a* and or b*, for example gray-blue, gray-green, and gray red. Dark colors typically have L<30, and typically are either saturated (a* and or b* not near zero) or unsaturated (a* or b* near zero which makes them heavily gray).

Reflectance spectra were experimentally obtained using a Data Color SF600SF Spectraflash color measurement system, calibrated with color standards traceable to the US NIST standards. A black trap was used to measure the E=0 value where L=0, a*=0, and b*=0. A US NIST traceable white reflectance standard was frequently measured to ensure proper instrument calibration.

In the experiments conducted for the present invention, reflectance measurements were made which included both the diffuse reflectance and the specular reflectance which allows calculation of L, a*, and b* for various surfaces. Diffuse reflectance occurs when light that is focused on a reflecting surface scatters in many directions as seen in flat colors and specular reflectance occurs when the angle of reflection equals the angle of incidence. When comparing the color and luminosity contrast between two different colored adjacent surfaces, it is useful to instrumentally calculate the function E of each surface from its L, a*, and b* values, where E²=L²+a*²+b*². The difference between the two E values (ΔE) for the two adjacent surfaces is used as a quantitative measure of the color differences (contrast) between these two surfaces where the lower the ΔE, the lower the contrast and visual perception of color differences. Correlations between ΔE values of the two adjacent surfaces and the contrast perception by typical observers, ranging from not observable (ΔE <3) to sharply contrasting (ΔE=50-100+), are shown in Table 1. When menstrual fluid is added to a white surface such as a white sanitary pad, there exists an extremely visible contrast between the red stain and the surrounding white surfaces resulting in high ΔE values, when the E values of the red stain are compared to the E value of the surrounding white surface measured individually. The objective of the current patent is to devise methods and devices that will lead to minimum hue, chromaticity and luminosity changes between a stain caused by deposition of menstrual fluid on the sanitary pad and the adjacent surfaces, so that the menstrual fluid on the pad will not or barely be noticed, as measured by low ΔE values. These ΔE values were instrumentally determined by measuring the E values of a candidate surface and then measuring the E value of this surface after being challenged by addition of artificial menstrual fluid.

TABLE 1
Correlation of ΔE values and Observer's
Perceptions

| Patterns | Solid Colors | |
|---|---|---|
| Scale for | Scale for | Observer's |
| ΔE | ΔE | Descriptions |
| <10 | <3 | Not observable |
| <20 | <10 | barely |
| | | observable |
| <30 | <20 | Low contrast |
| | | observable |
| <50 | <50 | Easily |
| | | Observable |
| 50 − 100+ | 50 − 100+ | Sharply |
| | | contrasting |

As a preliminary point, it should be understood that the red color of blood and menstrual fluid results from the red color of hemoglobin, which in blood is found within red blood cells but in menstrual fluid is believed to reside primarily in the dissolved state and not involve red blood cells. Some menstrual fluid simulants known and used in the prior art patent literature are composed of animal blood of various species that have been diluted approximately 50 percent with various water-based components. Similarly, the simulant used to acquire quantitative color masking data for the present invention was prepared by diluting decoagulated canine blood (treated to render it non-clotting) with distilled water in a 1:1 ratio and allowing this mixture to stand at ambient temperature at least two hours before use to lyse the red blood cells and release the hemoglobin into solution. This material is hereafter referred to as artificial menstrual fluid (AMF).

A typically configured feminine prior art sanitary pad 10 is shown in FIG. 1 looking downwardly on the optional topsheet 12. It should be appreciated that the prior art embodiment shown in FIG. 1 is illustrative in nature and not restrictive. Specifically, it should be appreciated that the color masking layer of the present invention may be used with essentially any configuration of feminine sanitary pad known in the art.

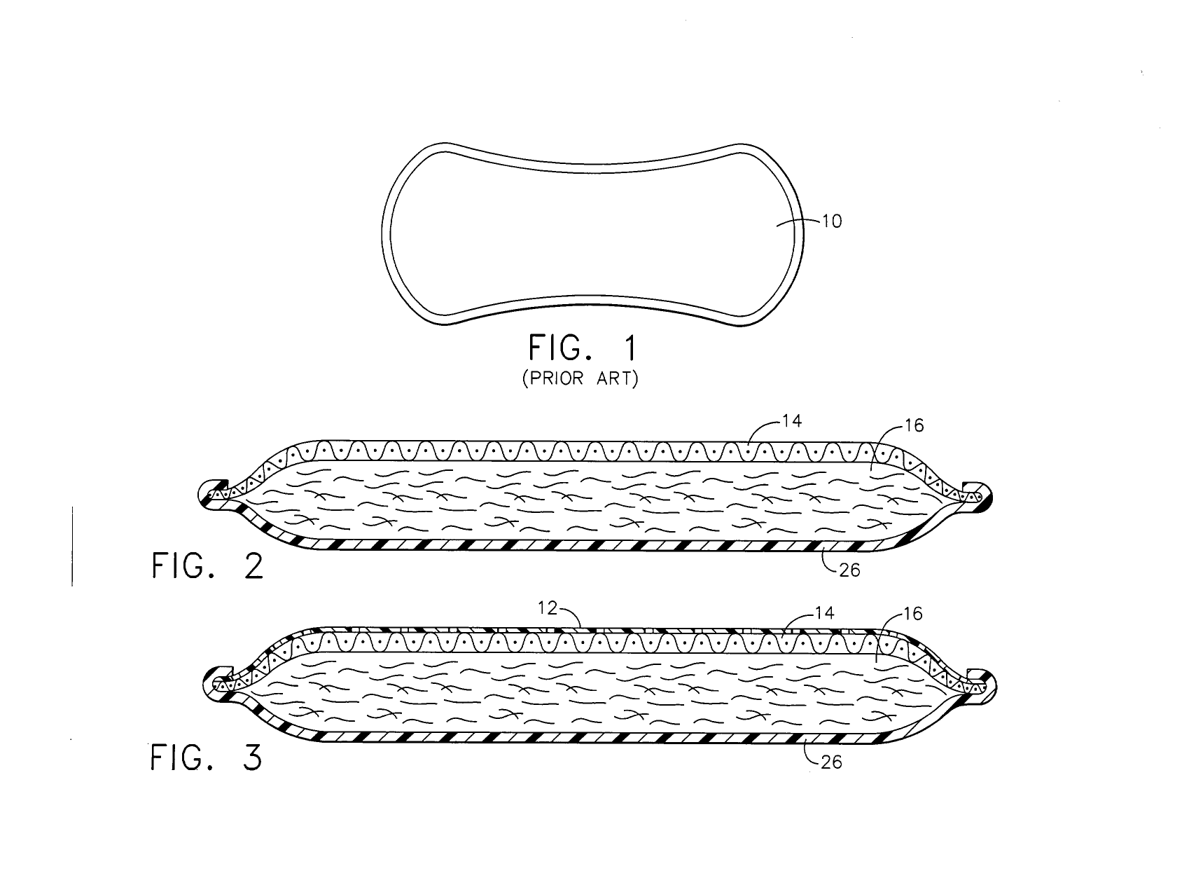

According to the present invention, and in order to reduce the visual perception of the presence of bodily fluid in the sanitary pad, a sanitary pad may be provided with a color masking layer. It should be appreciated that one or more of the layers of a typical pad's structure may be colored or rendered non-white so as to achieve the desired reduction in visual perception of staining. As shown in FIG. 2, one embodiment of such a pad structure incorporates a color masking layer 14 that includes a colored topsheet layer 12 which operates as both a typical sanitary pad topsheet as well as a color masking layer 14. Beneath the colored topsheet is disposed an absorbent core layer 16.( As shown In FIGS. 2-6, an impermeable backsheet 26 may be provided underneath the absorbent core 16.

Alternatively, and as shown in FIG. 3, a three layer pad structure may be incorporated in which a typical perforated topsheet 12 is disposed atop a colored underlayer color masking layer 14 which in turn is disposed atop the absorbent core 16. Perforated top sheets may have holes regularly or irregularly spaced, where holes or apertures are created by hole-punching or other means, or created by the manner in which the woven or non-woven topsheets are made.

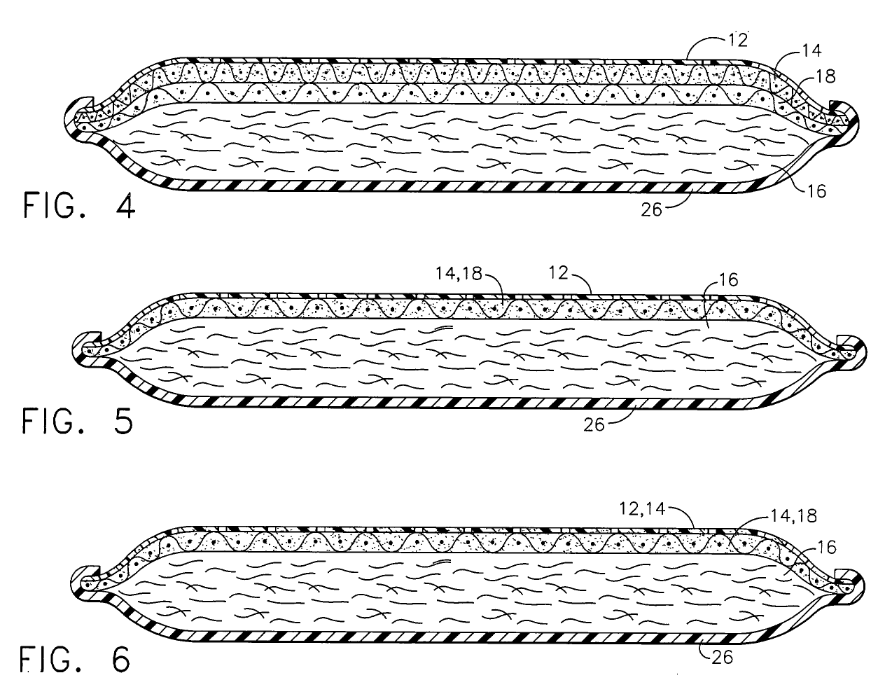

As shown in FIG. 4, a four layer pad structure may be incorporated in which a perforated topsheet (which is preferably hydrophobic to promote comfort and dryness) is disposed atop a colored under layer which acts as the color masking layer 14, which may optionally also serve as a containment layer. An optional spreading layer 18 is disposed between the color under layer 14 in the absorbent core layer 16.

Another alternate embodiment incorporating a color masking layer is shown in FIG. 5 wherein a perforated top sheet 12 is disposed atop a colored spreading layer 14, 18 which acts as the color masking layer and which is disposed top the absorbent core layer 16.

Finally, another potential illustrative embodiment is shown in FIG. 6 where the topsheet and spreading layer is colored and serves as a color masking layer and is disposed a top the absorbent layer 16. It should be appreciated that the embodiments shown in FIGS. 2-6 are only 5 examples of a sanitary pad provided with one or more color masking layers. It should be appreciated that it is within the scope of the present invention to provide any typical and known prior art sanitary pad with one or more color masking layers. The color masking layer may be a standalone hydrophobic colored layer or color may be imparted to a sanitary pad layer having additional functions other than color masking (i.e. absorbent core, topsheet, containment layer, spreading layer, and absorbent core).

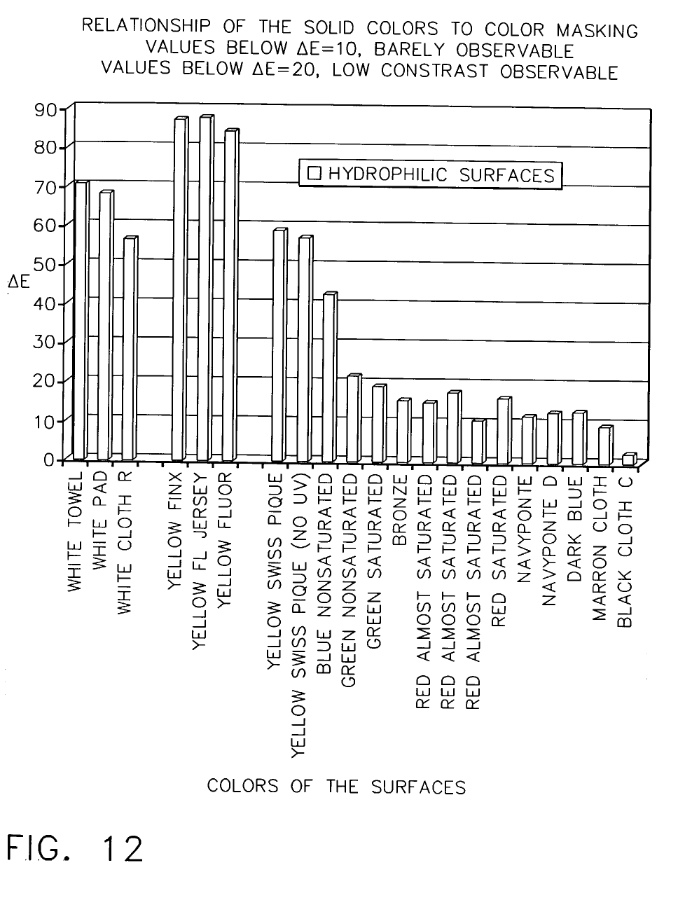

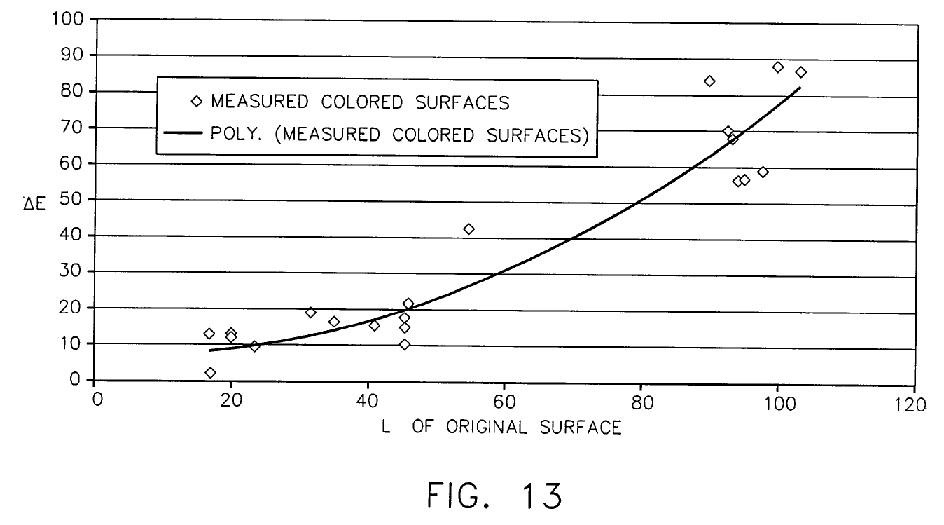

As shown in Table 2 and FIG. 12, various ΔE values (i.e. the difference between the unstained and the stained fabric E values) and L values for various experimental fabrics are provided. In FIG. 13, it can be seen that test results confirmed that fabrics with a relatively high degree of brightness and with high L values correspond to relatively high ΔE values as well. Furthermore, and as can be seen from the results in Table 2, it is clear that some colors and fabric compositions are better than others for reducing the visual perception of staining. At least some level of color masking typically occurs at ΔE values of less than 50 although some color masking can in fact occur at ΔE values between 50 and 70. It has been found that ΔE values less than or equal to 40 exhibit appreciable increase in color masking. As can be seen from the ΔE results in Table 2, the use of various colored cloths appreciably reduces the visual perception of staining as compared with white cloth or tissue. Although this is certainly an improvement realized by the incorporation of one or more colored layers, it should be appreciated that some consumers may desire additional reduction in color masking than is able to be provided by the use of colored sanitary pad layers as color masking layers alone.

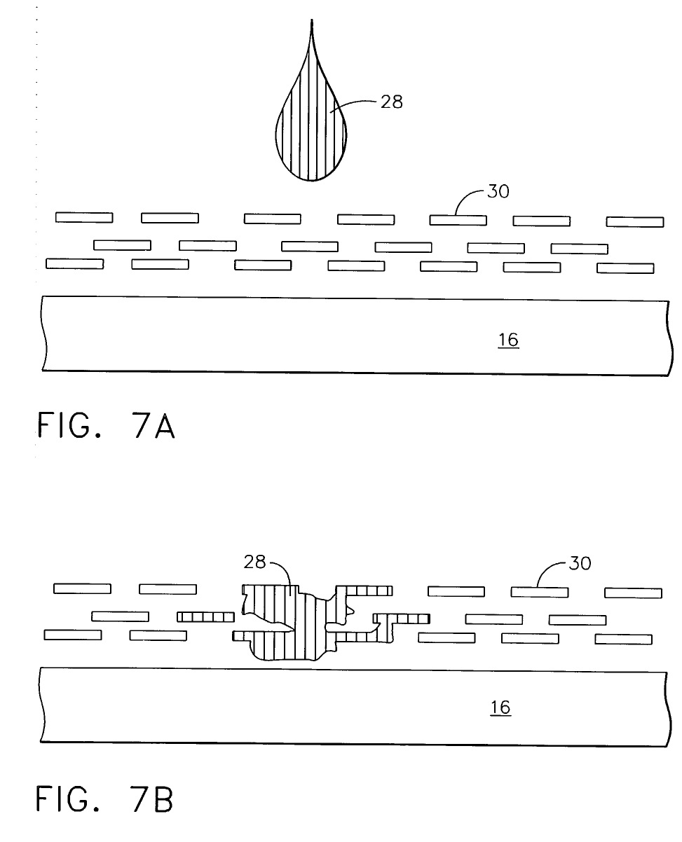

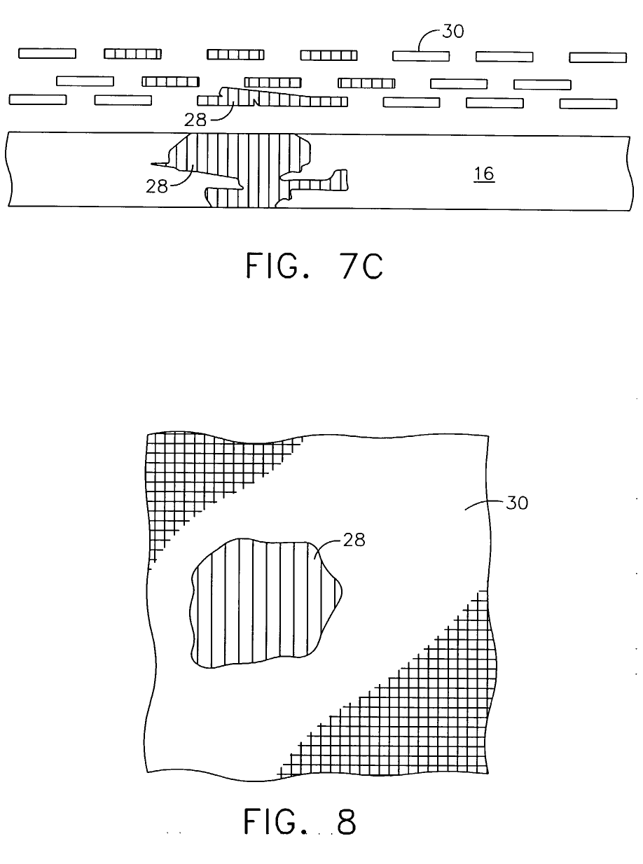

As best shown in FIGS. 7a-c and 8, simulations of the expected colors of a typical non-modified and commercially available sanitary pad equipped with a hydrophilic (thus wetable) containment layer fabric are diagrammatically and pictorially shown, before and after application of a simulated menstrual fluid 28. In FIG. 7a, before simulated menstrual fluid is applied to the pad, the pad appearance is white. As red blood or bodily fluid 28 comes into contact with the hydrophilic fabric of the containment layer 30 (FIG. 7b), the sanitary pad develops a stain and it is perceived as being red. Similarly, in FIGS. 7c and 8, as the fluid 28 is pulled down into the absorbent core 16, the pad clearly exhibits a red stain readily viewable by an observer due to seeing the residual fluid within the woven fabric 30 and potentially on the absorbent core (depending on the containment layer weave tightness).

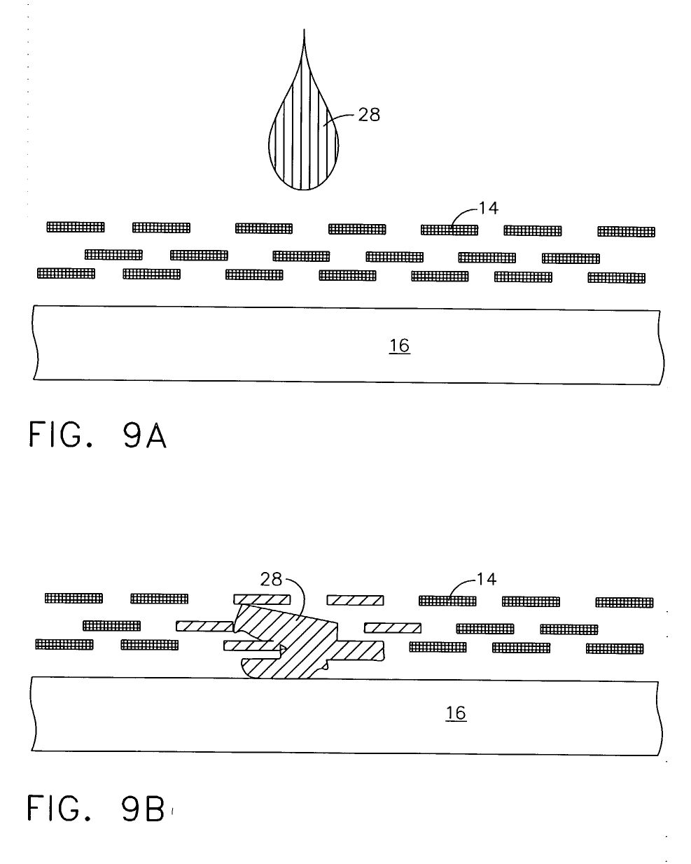

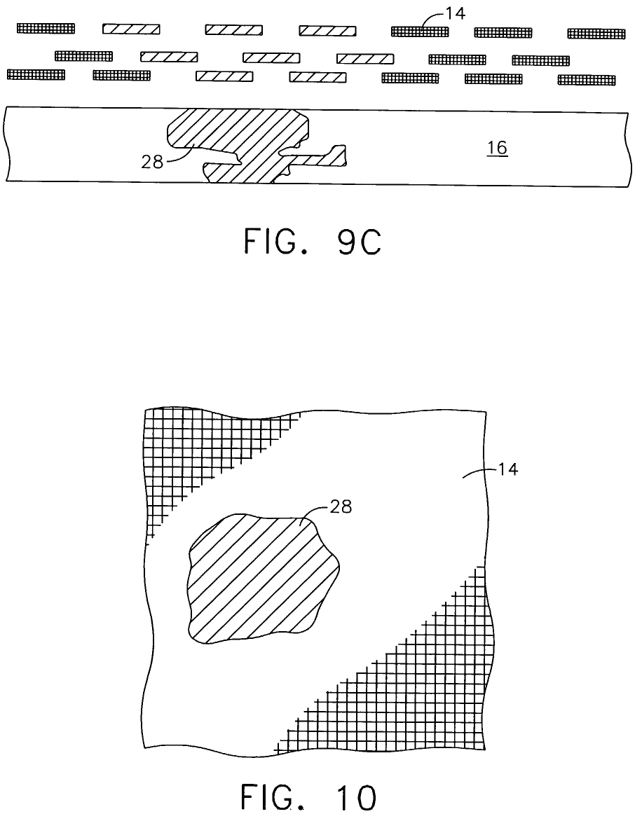

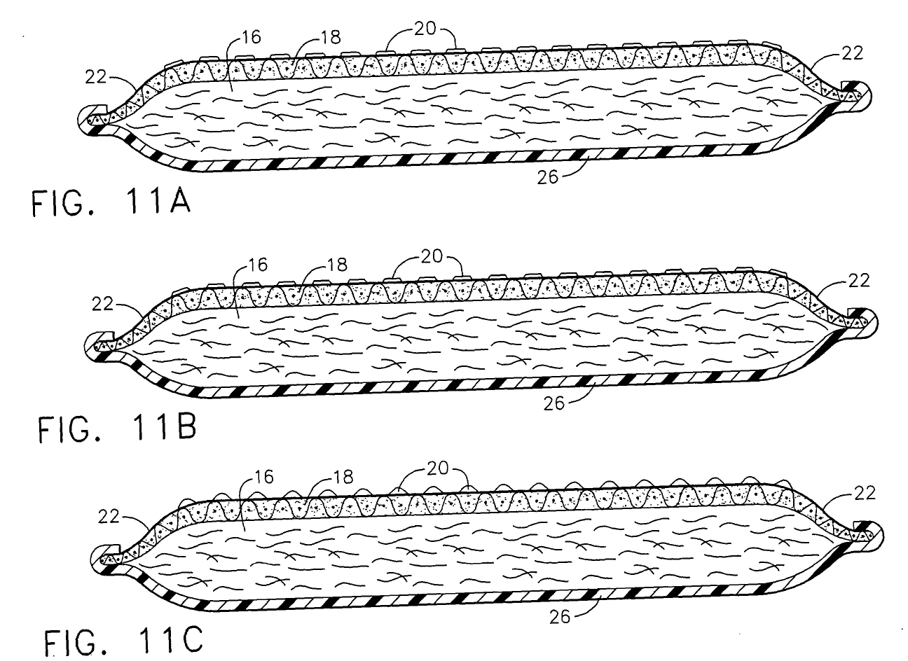

It is one goal of the present invention to minimize the visual perception of a stained sanitary pad by incorporating one or more color masking layers in the construction of the pad. To illustrate this, and as shown in the color simulations in FIGS. 9a-c and 10, when the hydrophilic (thus wetable) containment layer fabric 14 is dyed a color other than white, there is less visual contrast between the original fabric color and the red color of the fluid 28. Accordingly, there is not as much color contrast between the stained and unstained areas by an observer as compared with the contrast between the stained and unstained areas of a standard unimproved white pad. This reduction in the visual perception of the stain is desirable. Although the visual perception of the staining is clearly reduced, it can be seen in the simulated figures that staining is still somewhat observable by an observer. Accordingly, as mentioned previously, some users may prefer further reduction in the visual perception of staining than available by the incorporation of a non-white color masking layer into a sanitary pad.

TABLE 2
ΔE and L values of colored fabrics after application of AMF

| | | Original |
|---|---|---|
| | | Brightness |
| Sample | Delta E | L Value |
| White Facial Tissue 1 | 70.38 | 92.36 |
| White Facial Tissue 2 | 68.16 | 92.93 |
| White Cloth^{R} | 56.52 | 93.87 |
| Yellow FI-Jersey | 86.85 | 103.07 |
| Yellow FL Jersey | 87.74 | 99.62 |
| Yellow Fluor | 84.1 | 89.67 |
| Yellow Swiss Pique | 59.05 | 97.47 |
| Yellow Swiss Pique (no UV) | 57.08 | 94.71 |
| Blue Non-saturated | 42.55 | 54.68 |
| Green Non-Saturated | 21.74 | 45.78 |
| Green Saturated | 19.18 | 31.64 |
| Bronze | 15.54 | 37.9 |
| Red Almost Saturated | 15.11 | 45.41 |
| Red Almost Saturated | 17.57 | 45.42 |
| Red Almost Saturated | 10.35 | 45.41 |
| Red Saturated | 16.33 | 34.95 |
| Navy Ponte | 11.91 | 19.93 |
| Navy Ponte D | 12.79 | 19.85 |
| Navy Blue | 13.01 | 16.87 |
| Maroon | 9.34 | 23.31 |
| Black C | 2.36 | 17 |

TABLE 3
Topsheets on Fabrics after Insult with Artificial
Menstrual Fluid (AMF)

| Sample Designation | | | |
|---|---|---|---|
| (Colored Topsheet | | | |
| on Colored Fabric) | Topsheet L | ΔE | Observations |
| Orange Glo TredHexPent | 71 | 6.85 | Barely |
| On Black | | | observable |
| Shy Blue HexPent | 70 | 8.1 | Barely |
| On Black | | | observable |
| Orange Tred22Hex on Red | 59 | 5.84 | Barely |
| | | | observable |
| No Topsheet/Red | — | 19.95 | Observable |
| Orange Tred22hex on Blue | 59 | 4.17 | Barely |
| | | | observable |
| No Topsheet/Blue | — | 26.78 | Easily |
| | | | observable |

It should be appreciated that as the color of the hydrophilic (thus wetable) containment layer fabric more closely approximates the red color of the bodily fluid, there is even less contrast to be observed by the user. In this case, the contrast between the colored stain on the colored fabric is not as great as the contrast between the colored stain on white fabric of the prior example. Nonetheless, it is clear that at least some staining is visible and additional color masking would be greatly preferred compared to that which may be accomplished by use of hydrophilic colored containment layer fabric alone. Obviously, if the fabric of hydrophilic containment layers were to be red or substantially dark, or black in color, very little of the stain would be apparent. However, the degree of observable staining of different colored fabrics where the ΔE values is about 40 or below could be acceptable to a portion of the user community. Furthermore it was shown that placing white or colored topsheets on top of various colored cloths gave rise to increased color masking as evidenced by decreased ΔE values when a topsheet was used compared to not using a topsheet. Furthermore, as mentioned previously and as shown in Table 3, it was shown that increased color masking was achieved when orange or blue topsheets were used compared to white topsheets as indicated by further reductions in ΔE values. Nonetheless, it is thought that this moderate degree of color masking would not have widespread acceptance in the marketplace and hence, other color masking approaches and materials, besides the use of colored containment layer fabrics, is needed to provide an acceptable level of color masking while still providing a sanitary pad that has an appealing appearance, functions at least as well as non-modified pads, and is commercially acceptable.

In order to truly facilitate and enable color masking of menstrual fluid as well as other bodily fluids, one embodiment of the present invention including providing a color masking layer of small, hydrophobic, colored (including white) surfaces on a colored (preferably dark) substrate of hydrophilic woven or non-woven fabric (also referred to as cloth) comprising the containment layer. A hydrophobic material can be defined as one that gives high contact angles when water is placed on its surface and the higher the contact angle the greater the hydrophobicity of that material. In general, the higher the contact angle, the greater the tendency for a drop of water to roll off a surface as this surface is tilted from being horizontal to the earth's surface. Thus, those materials with increased hydrophobicity will be expected to have increasing application for preparation of such hydrophobic surfaces. The surfaces of these hydrophobic surfaces will also be impermeable towards water or aqueous-based solutions. Hereafter, the term liquid impermeable will be used to refer to a material that is impermeable to water or aqueous-based solutions.

Preferably, this layer of hydrophobic, liquid impermeable colored surfaces is disposed atop this containment layer and attached thereon by suitable film deposition or printing process means known in the art, such as silk screen printing, transfer printing, stipple printing, coating processes, and use of adhesives to anchor such surfaces. Although the phrase colored surface is used herein, it should be appreciated that the shape of the hydrophobic materials may be varied in size, spacing, color, pattern, color brightness, reflectance, and opacity without departing from the present invention. Substantially round colored surfaces are described and shown herein as one preferred embodiment of the present invention. Another embodiment of the present invention incorporates narrow colored (including white) ribbons of hydrophobic and fluid impermeable material disposed on a colored (preferably dark) substrate of hydrophilic woven or non-woven fabric comprising the containment layer.

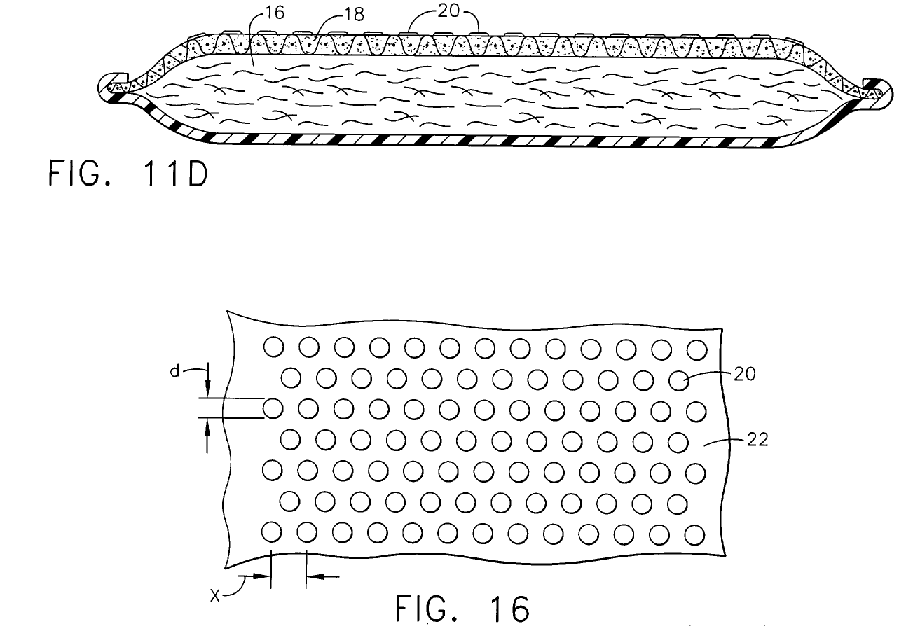

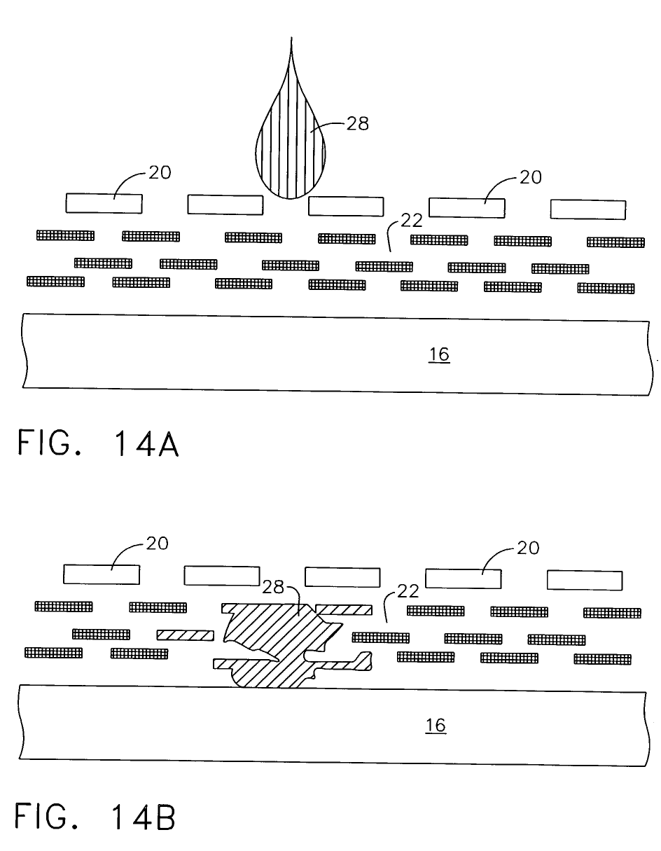

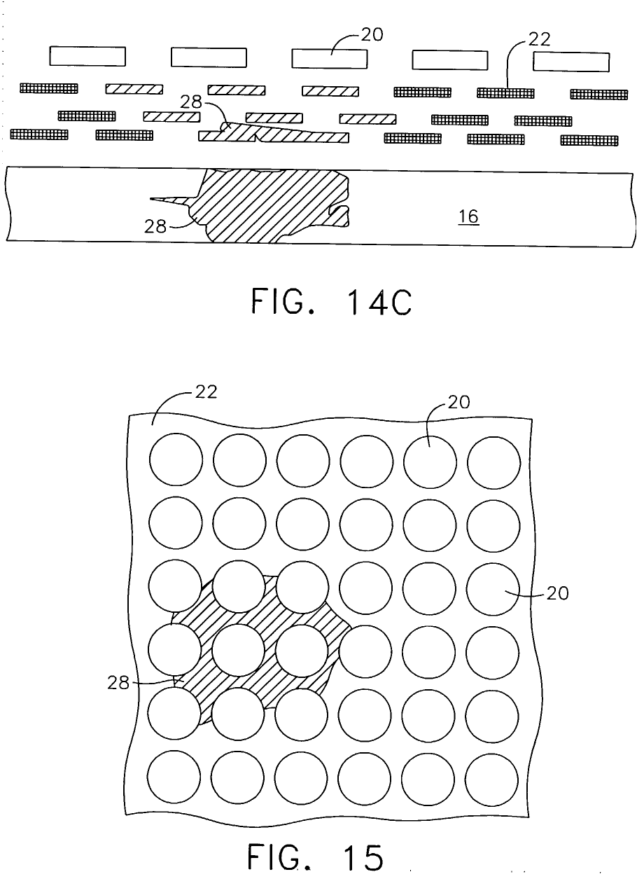

To better illustrate how the color masking layer of the present invention incorporating a plurality of hydrophobic disks works reference is made to FIGS. 11a-d which show layers of support fabric 22 supporting a plurality of hydrophic surfaces 20. Hydrophobic and fluid impermeable surfaces 20, as seen in FIGS. 11a-c, are preferably of any suitable configuration that separates the user from the underlying support fabric. The surfaces 20 are preferably flat, beveled (FIG. 11b), bubble-shaped, conical (FIG. 11c) or essentially of any configuration that promotes fluid removal from the surfaces. As shown in FIG. 11d, the surfaces 20 may be disposed directly on a spreading layer 18 instead of a separate support fabric 22. FIGS. 14a-c pictorially simulates a plurality of substantially uniformly spaced colored hydrophobic surfaces 20 supported on a dark substrate of hydrophilic woven or non-woven fabric 22 (which is also the fabric of the containment layer). As shown in FIGS. 14b and 14c and 15, the bodily fluid 28 is not retained on the hydrophobic and fluid impermeable surfaces 20 and is drawn between the spacing of the hydrophobic surfaces 20 by wicking into the preferably hydrophilic fabric of the containment layer 22 from which it is transferred into the absorbent core 16.

A primary reason that the red color of the menstrual fluid or its stain is not visible or is only barely visible is that the high reflected light intensities from the colored hydrophobic surfaces hide the slight darkening of the fabric support layer exposed between the colored surfaces from visual detection. Use of fluorescent or reflective pigments or reflective backings on the colored surfaces will further aid the color masking process by enhancing the light intensities emanating from the colored surfaces, while diminishing even further the perception of the slight staining of the dark colored support fabric. Thus, when the hydrophobic colored disks are used in conjunction with dark colored support fabric, the color masking capabilities of the invention are increased.

Following are the operative principles that cause this surface to appear essentially the same before and after application of artificial menstrual fluid: (1) The fluid is effectively shed from the hydrophobic, fluid impermeable colored surfaces and wicks into the wettable hydrophilic (wettable) support fabric. (2) The black color of the support fabric undergoes a slight change of color or shade but visual observation of the actual color or shade of the support fabric is barely perceived due to the high light intensity reflected from the colored surfaces arranged on the dark-colored support fabric. (3) Visual obscuration of the dark-colored support fabric may be enhanced by the desired high light reflectivity of each colored surface due to the fact that these color surfaces may be backed with a very thin reflective white or metallic layer. An alternate method to achieve high reflectivity in color surfaces of any color is to use fluorescent pigments or dyes or mix highly reflective materials in the colored surface pigment or dye. It has been demonstrated that the range of open space area to colored surface area can range from less than 10% to greater than 95% while achieving effective color masking and efficient menstrual fluid transfer to the lower layer

The support fabric for hydrophobic colored ribbons and color surfaces are preferably dark colored so that absorbed bodily fluids are not apparent. Similarly, good wicking or wetting properties of the support are also highly desirable features. Opaque fibers with high wetability may be desirable as support fabric for colored ribbons and color surfaces used in color masking of menstrual fluid.

The color masking layer of the present invention may also be provided with indicator windows or “non-covered” or “non-masked” areas at various locations on the pad to indicate to the wearer the relative remaining absorption capacity. The windows may simply turn red to indicate the presence of menstrual fluid, or a reagent may be used to signal the amount of fluid being retained in the pad. Alternatively, a reagent may be used to turn a different color (other than red) to signify the presence of fluid in the pad and indicate the remaining capacity of the pad, based on the extent that these windows positioned at increasing distance from the center of the pad were colored the appropriate indication color. In addition, the “windows” may comprise decorative patterns, pictures, sayings, or logos whereby migrating menstrual fluid could supply specific regions of color to a pattern or picture. Additionally, patterns, emblems, insignias, pictures, landscapes, slogans and the like may be superimposed or printed on the whole or part of the topsheet or color masking layer of the present invention to complement the colored feminine hygiene pad surfaces to make menstrual fluid stains less noticeable in accordance with the principles of color masking described herein. Such visual features listed above can contribute to the color masking effects primarily contributed by the use of hydrophobic colored surfaces or colored ribbons by providing visual distraction to the pad user, both before and after use. Such additional features may be supplied by using any of the colors available within the visible spectrum, including white and black, and such colors may be generated using non-reflective, reflective, and(or) fluorescent pigments. More than one color or material may be presented by the hydrophobic surfaces so that patterns may be established across a number of such surfaces to create the various effects discussed above.

The main function of a topsheet is to provide a hydrophobic and non-wettable surface that will separate the body of the user from the pad containment layer and maintain a relatively dry feeling. As such, the hydrophobic and liquid impermeable surface structure may also serve the role of a topsheet when these hydrophobic surfaces are either flush with the support fabric or are raised above the support fabric, since this geometry will serve to separate the skin from the color masking support layer (which may also be serving as the containment layer). It could be expected that the hydrophobic surfaces could most effectively serve the additional role of serving as a topsheet when these surfaces are raised above the surface of the hydrophobic layer to provide increased spacing between the body and the support fabric. In this case having rounded or beveled edges would be an important structural feature to incorporate not only for enhanced shedding of menstrual fluid but also for enhanced wearer comfort. This feature would provide an inherent economic advantage to the manufacturer in that an additional topsheet layer may not be needed to provide optimal dryness to the user.

Rapid Flow Support Fabrics for Hydrophobic Surfaces. As discussed above, one embodiment of the present invention for color masking devices involves attachment of colored hydrophobic and liquid impermeable surfaces to a hydrophilic support fabric. This support fabric also serves to transport menstrual fluid, blood, or urine released to the colored hydrophobic surfaces to the absorptive core positioned below the support fabric. Described herein is the use of various sized hydrophobic surfaces with various spacings between these surfaces wherein the percent covered area of the support fabric ranges between about 10 percent up to about 95 percent. Accordingly, in order for these described devices to overcome the effect of this coverage and to absorb and transport released menstrual fluid, blood, or urine at sufficiently high permeation rates to the underlying absorptive core, it will be advantageous to use support fabrics that are readily wetted and have high permeation rates towards these and other water-based fluids. However, it is known that economical support fabric candidates such as nylons, polyesters, acrylics and other polymers may be relatively hydrophobic and become wetted and transport water at low to moderate flow rates. Hydrophilic fabrics such as cotton have high fluid capacity but do not have high permeability rates since the water is held tightly. One method used in the apparel industry to enhance wicking and permeation rates of water on relatively hydrophobic fabrics is to coat these fibers with hydrophilic components that may or may not be covalently attached to the fiber hydrophobic core. Another method is that the fibers of the support fabric may consist of bundles of very small diameter fibers to increase the effective fiber surface area and thus enhance fluid migration rates along such fibers. Without wishing to endorse specific products, the following fabrics employ this technology and were determined to have high transport properties towards menstrual fluid simulants, thus making them appropriate for use in the devices described herein: Intera® I-301 and I-303 by Intera Technologies Inc. (coating on nylon), inteX GB 2821, Xhale™ by Intex Corp. (coating on polyester), Synatural™ Fabrics with Nano-dry™ by Burlington Raeford (coating on polyester), and hydrophilic coating on nonwoven spunbonded polypropylene by Mogul. Fabrics that have modified weaving patterns designed for enhanced liquid transport properties or specially shaped fibers such as CoolMax® by DuPont also are good candidates for rapid flow support fabrics. Another method to enhance the permeation rate of support fabrics towards aqueous solutions is to coat such fabrics with hydrophilic surfactants such as Lurol PP-9725 by Goulston Technologies. Candidate support fabrics for attachment of hydrophobic surfaces include woven, knitted, and nonwoven fabrics.

Where the color masking layer is directly contacts the user, it has been found that fabrics having fluid impermeable or hydrophobic areas extending upward toward the user advantageously provide comfort to the wearer as the greater the height of the areas, the further the user's surface from the wetted support fabric of the color masking layer. While there is a limit to this height, it may be understood that the height of the hydrophobic material on the color masking layer may be optimized to enhance comfort when directly in contact with the user. Further in this regard, the shape of the hydrophobic area may further be optimized to provide smooth surfaces, and angled peripheral surfaces to enhance comfort and promote rapid flow of fluids into the absorbent article.

Deposition of Experimental Array of Hydrophobic Surfaces on Support Fabrics. To quantify the effects of various hydrophobic surfaces on various support fabrics, an experimental array of differently colored hydrophobic surfaces composed of polyvinyl chloride (PVC) were deposited upon differently colored fabrics and were primarily used to experimentally determine those combinations of surface color, support fabric color, surface size, and surface separations that resulted in the most effective color masking. As shown in FIG. 16 (not to scale), these surfaces 20 were prepared from thermally set PVC plastisols by screen printing and had nominal diameters d of 0.3 mm, 0.5 mm, 1.0 mm, and 2.0 mm were deposited by silk screening on support fabrics with nominal separation distances x (distance of closest approach) of 0.1 mm, 0.2 mm, 0.5 mm, 1.0 mm, and 2.0 mm after a final heat setting step. This approach resulted in hydrophobic surfaces that were essentially flush with the fabric support layer and thus could also serve as a topsheet as mentioned previously. For purposes of this testing, and as shown in the exemplary array of surfaces depicted in FIG. 16, the surfaces may be disposed in alternating staggered rows such that surfaces in alternate rows are substantially aligned.

PVC is a relatively hydrophobic and liquid impermeable polymer that sheds water and was expected to effectively shed artificial menstrual fluid derived from diluted canine blood. However, it was determined that some PVC pigments, which may migrate to the surface of hydrophobic polymers, were apparently sufficiently hydrophilic to cause partial retention of the red color of hemoglobin-based simulated menstrual fluid. One approach to the problem is to increase the overall hydrophobicity of PVC surfaces by mixing in various quantities of a highly fluorinated polymer (such as Cytonix FluorPel™ PFC 1602A) within the PVC plastisol and this approach was found to give some enhanced properties. Alternatively, the entire hydrophobic surface may be composed of a fluoropolymer or other material with enhanced hydrophobicity. Hydrophobic surfaces composed entirely of pigmented fluoropolymer (Cytonix Q348 PerFluoroCoat™) were deposited on support fabric (Series α and β) and found to have no observable retention of red color derived from the artificial menstrual fluid and also gave among the lowest ΔE values that were measured in the experimental matrix. It was also determined that another method to significantly enhance the aqueous solution permeation rate of artificial menstrual fluid through color masking layers was to spray the entire color masking layer consisting of hydrophobic surfaces on support fabrics with a hydrophilic surfactant such as Lurol PP-9725 by Goulston Technologies. This approach led to significantly increased fluid permeation rates through the entire color masking layer without decreasing the color masking effect (See ΔE data for series α and β for discs prepared there from).

Test systems of hydrophobic surfaces on support fabrics were also effectively produced with the Chromatec Color System, which involves transfer of a desired pattern from a mask by photochemically curing a pigmented layer behind this mask and adhesively attaching the generated surface pattern to support fabrics. Use of the Chromatec Imaging System allowed formulation of differently colored fluorescent pigments that resulted in a high degree of color masking. It was also determined that placing a white layer immediately beneath the pigmented layer gave significantly enhanced color intensity that contributed to the color masking effect. The Chromatec approach was used to produce hydrophobic surfaces that were raised above the fabric support layer and these surfaces also had beveled edges. As mentioned, the use of hydrophobic surfaces that were raised above the support fabric could enhance their role as serving as built-in topsheets.

In order to assess color masking capabilities, various commercially available modified fabrics that had color masking potential were obtained and evaluated. One type modified fabric has a regular array of relatively closely spaced hydrophobic surfaces that were attached to support fabric. In two such cases (Gold Lame 8-8 and 8-12), it was determined that the fabric consisted of bundles of small diameter polyethylene terephthalate fibers and the surfaces had a sandwich structure composed of an upper pigmented polyurethane layer positioned above a highly reflective layer that was positioned above a lower thermoplastic polyurethane presumably used for thermal attachment to the underlying fabric Another type commercially available modified fabric has narrow reflective and colored ribbons that were arranged in parallel rows and attached to the support fabric. Another type fabric had small sized discs that were coated with multiple colors and had superimposed interference patterns and(or) hologram patterns superimposed on the entire disc system. Other reflective surfaces are useful, such as Moire' patterns, foils, fluorescent pigments and dyes and other reflective materials deposited in layers.

A test matrix was evaluated where artificial menstrual fluid was added to hydrophobic disc systems composed of polyvinyl chloride attached to support fabrics by screen printing and their reflectance behavior was measured instrumentally. Each sample was placed above a spreading layers which was disposed above absorptive paper and artificial menstrual fluid (0.5 mL) was added evenly over a 1.7 cm diameter region. A heavy washer having a 1.7 cm diameter hole was placed over the hydrophobic disc systems to focus addition of the artificial menstrual fluid within this desired area. These hydrophobic disc systems had a variety of colors and had a range of sizes (0.3 mm to 2.0 mm in diameter) and different spacings. Hydrophobic surface colors were chosen to have a lighter color and higher luminosity than the support fabric luminosity and color so that when red menstrual fluid enters the support fabric, a minimal overall color and luminance color change will be observed. It was expected that the darker the fabric the lower the observed color and luminosity change when red menstrual fluid simulant was absorbed by the fabric. Hydrophobic surfaces having light colors and high luminosities will overpower the eye's perception of the adjacent support fabric having darker colors and low luminosities so that the human perception of the device is mainly that of the lighter colored hydrophobic surfaces before and after the addition of menstrual fluid. An exception to this effect occurs when red menstrual fluid is added to the range of red fabrics wherein both the color and luminance changes will be small (color matching rather than color masking). It was expected that as the luminance of the support fabric increased (become less black) color masking would become less efficient as measured by visual perception and an increased ΔE value. These principles were tested by placing hydrophobic surfaces with a range of colors on support fabrics ranging from black (having very low luminance) to white (having high luminocity).

One “observed” benefit of high hydrophobic surface reflectivity or luminosity (or high L value) is that it renders the color of dark support fabrics barely evident to the observer as evidenced by the following laminated systems having significant percent open area: gold surfaces on polyester (gold lame' 8-8; 54% open area) and Series C, 0.5 mm×0.5 mm); white surfaces on bronze fabric (series P, 0.5 mm×0.5 mm in particular with 50% open area; a wide range of green foil surface sizes and separations (series X) had a number of surfaces appearing to be completely covered while performing excellently in not retaining artificial menstrual fluid.)

Some of the test results and data generated are shown in Table 4. Specifically, Table 4 summarizes, both objectively and by visual observation, some of the results of this testing including hydrophobic gold foil, hydrophobic white surfaces, hydrophobic green foil surfaces and hydrophobic green fluoropolymer surfaces all on black support fabrics. Additionally, Table 4 shows the results of various sized hydrophobic red surfaces on blue support fabric as well as Chromatec green surfaces on bronze support fabrics. The subjective evaluations are observations by the naked eye, at a distance of approximately one foot or greater.

TABLE 4
Examples of ΔE Data on Some Preferred Hydrophobic Surface/Support Fabric
Systems

| | Hydrophobic | Percent | | | Observations |
|---|---|---|---|---|---|
| | Diameter (area) | Closed | | System | of Challenge |
| Sample | mm (mm²) | Area | ΔE | L Value | Result |
| Gold Lame on | | | | | |
| Black | | | | | |
| Gold Lame 8-8-a1 | 0.6 (0.3) | 50 | 4.3 | 68.9 | Barely Observed |
| Gold Lame 8-8-a1 | 0.65 (0.3) | 50 | 2.4 | 68.9 | Barely Observed |
| Gold Lame 8-8-a1 | 0.6 (0.3) | 50 | 2.9 | 68.9 | Barely Observed |
| Gold Lame 8-8-a2 | 0.6 (0.3) | 50 | 3.5 | 70.0 | Barely Observed |
| Gold Lame 8-12 | 0.6 (0.3) | 40 | 4.0 | 59.9 | Barely Observed |
| Gold Foil Black C | 0.3 (0.07) | 40 | 1.4 | 54.9 | Barely Observed |
| Gold Foil Black C | 0.3 (0.07) | 20 | 2.9 | 37.4 | Barely Observed |
| Gold Foil Black C | 0.3 (0.07) | 5 | 2.9 | 27.5 | Barely Observed |
| Gold Foil Black C | 0.3 (0.07) | 2 | 3.2 | 19.8 | Barely Observed |
| Gold Foil Black C | 0.5 (0.2) | 75 | 4.7 | 73.6 | Barely Observed |
| Gold Foil Black C | 0.5 (0.2) | 50 | 1.8 | 66.3 | Not observed |
| Gold Foil Black C | 0.5 (0.2) | 50 | 1.9 | 66.3 | Not Observed |
| Gold Foil Black C | 0.5 (0.2) | 15 | 3.1 | 40.4 | barely observable |
| Gold Foil Black C | 1 (0.8) | 80 | 3.5 | 76.3 | barely observable |
| Gold Foil Black C | 1 (0.8) | 80 | 4.2 | 76.4 | barely observable |
| Gold Foil Black C | 1 (0.8) | 60 | 5.0 | 66.3 | barely observable |
| Gold Foil Black C | 1 (0.8) | 25 | 3.6 | 48.4 | barely observable |
| Gold Foil Black C | 1 (0.8) | 80 | 3.5 | 76.3 | barely observable |
| Gold Foil Black C | 1 (0.8) | 80 | 4.2 | 76.4 | barely observable |
| Gold Foil Black C | 1 (0.8) | 60 | 4.9 | 66.3 | barely observable |
| Gold Foil Black C | 1 (0.8) | 25 | 3.6 | 48.4 | barely observable |
| Gold Foil Black C | 2 (3.1) | 70 | 4.4 | 73.3 | barely observable, residue |
| Gold Foil Black C | 2 (3.1) | 60 | 3.1 | 69.36 | barely observable residue |
| Gold Foil Black C | 2 (3.1) | 40 | 1.4 | 59.7 | barely observable, residue |
| Gold Foil Black C | 2 (3.1) | 40 | 0.84 | 59.4 | barely observable |
| White on Black Series | | | | | |
| White black Series B | 0.3 (0.07) | 40 | 1.6 | 42.5 | barely observed, wet only |
| White black Series B | 0.3 (0.07) | 20 | 1.3 | 31.6 | barely observed, wet only |
| White black Series B | 0.3 (0.07) | 5 | 1.4 | 21.9 | barely observed, wet only |
| White black Series B | 0.5 (0.2) | 75 | 1.9 | 62.4 | barely observed, wet only |
| White black Series B | 0.5 (0.2) | 50 | 2.5 | 55.3 | barely observed, wet only |
| White black Series B | 0.5 (0.2) | 15 | 1.5 | 33.3 | barely observed, wet only |
| White black Series B | 1 (0.8) | 80 | 3.9 | 69.7 | barely observed, some |
| | | | | | red in dot center |
| White black Series B | 1 (0.8) | 60 | 2.8 | 63.1 | barely observed, some |
| | | | | | red in dot center |
| White black Series Br | 1 (0.8) | 60 | 2.2 | 63.1 | barely observed, some |
| | | | | | red in dot center |
| White black Series B | 1 (0.8) | 25 | 3.0 | 43.7 | barely observed, some |
| | | | | | red in dot center |
| White black Series B | 2 (3.1) | 70 | 6.7 | 70.7 | low contrast observable, |
| White black Series Ba | 2 (3.1) | 70 | 8.5 | 70.7 | low contrast observable, |
| | | | | | some red in dot center |
| White black Series B | 2 (3.1) | 60 | 3.7 | 63.7 | barely observable, some |
| | | | | | red in dot center |
| White black Series Ba | 2 (3.1) | 60 | 4.5 | 63.7 | barely observable, some |
| | | | | | red in dot center |
| White black Series B | 2 (3.1) | 20 | 1.9 | 50.5 | barely observable |
| Green Foil on Black | | | | | |
| Green Foil-Black Series X | 0.3 (0.07) | 40 | 6.68 | 41.6 | not observable |
| Green Foil-Black Series X | 0.5 (0.2) | 75 | 5.4 | 51.6 | not observable |
| Green Foil-Black Series X | 0.5 (0.2) | 50 | 3.9 | 45.3 | not observable |
| Green Foil-Black Series X | 1 (0.8) | 60 | 2.2 | 43.8 | not observable |
| Green Fluoropolymer on Black | | | | | |
| GreenFluoropolymer-Black | 1 (0.8) | 80 | 0.7 | 33.2 | not observable, wet |
| Series α | | | | | |
| GreenFluoropolymer-Black Surf. | 1 (0.8) | 80 | 2.0 | 33.1 | not observable, wet |
| Series α | | | | | |
| GreenFluoropolymer-Black | 1 (0.8) | 60 | 0.65 | 30.8 | not observable, wet |
| Series α | | | | | |
| GreenFluoropolymer-Black. | 1 (0.8) | 80 | 1.61 | 33.2. | not observable, wet |
| Series .β | | | | | |
| GreenFluoropolymer-Black Surf. | 1 (0.8) | 80 | 1.10 | 33.6. | not observable, wet |
| Series .β | | | | | |
| GreenFluoropolymer-Black. | 1 (0.8) | 60 | 2.08 | 30.4. | not observable, wet |
| Series .β | | | | | |
| Red on Blue | | | | | |
| Red-Blue Series L | 0.5 (0.2) | 75 | 3.5 | 31.0 | Barely observable |
| Red-Blue Series L | 0.5 (0.2) | 50 | 2.5 | 30.0 | Barely observable |
| Red-Blue Series L | 1 (0.8) | 80 | 1.6 | 32.3 | Barely observable |
| Red-Blue Series L | 1 (0.8) | 60 | 2.6 | 32.2 | Barely observable |
| Chromatec Green on Bronze | | | | | |
| Chromatec Green-Bronze | 0.65 (1.4) | 70 | 4.5 | 68.2 | barely observable |

It was generally observed that the artificial menstrual fluid to these systems rapidly permeated within seconds through the color masking layers to the underlying surfaces. However, the 0.5 mm and 1.0 mm discs having approximately 75% closed area and 80% closed areas, respectively, had relatively slow permeation rates due to the low area percentage of liquid-permeable hydrophilic fabric surface. However, it was observed in these cases that these slow permeation rates could be markedly increased by spraying the entire color masking layer (colored hydrophobic discs attached to support fabrics) such as Lurol PP-9725 hydrophilic surfactant while allowing to try before testing. Without wishing to be bound by theory, it is thought that hydrophilic surfactant is effectively shed from the hydrophobic surfaces and migrates to the support fabric wherein the fluid migration properties of the support fabric are increased. This increase in permeation was noted and measured in several systems such as in the alpha and beta series (green Cytonix Q348 PerFluoroCoat™ where the beta series, after screen printing, involved a final hot press but the alpha series did not). In the alpha series, the 1.0 mm discs with approximately 80% closed area retained the artificial menstrual fluid for about 60 seconds after which the AMF rapidly moved though the color masking layer. However, after treating with this hydrophilic surfactant, the AMF was observed to immediately permeate through the color masking layer. This type behavior was also repeated in the beta series where discs having the same dimensions specified for the alpha series retained the AMF for about 40 seconds but after these were treated with hydrophilic surfactant, the AMF was observed to immediately permeate through the color masking layer. This effect was noted in a commercially obtained material having closely spaced discs on a support fabric (Foxy Spandex) whereby there was a 60 second delay before AMF placed on its surface permeated the color masking layer whereas the AMF permeated through almost immediately after treating with hydrophilic surfactant.

A beneficial effect of using a mixture of a fluoropolymer and PVC was also noted when hydrophobic discs were composed of 0.3 percent FluorPel™ Cytonix 1602A in PVC that contained a yellow pigment (Series T on dark blue fabric) and this system was compared to the same system without FluorPel™ Cytonix 1602A (Series F on dark blue fabric) when the disc had 1.0 mm diameters and separations of approximately 0.5 mm. It was found that the discs containing FluorPel™ Cytonix 1602A gave a “very light” stain with no red dots present whereas the discs not containing FluorPel™ Cytonix 1602A gave a residue with a “light” stain with small red dots derived from the AMF present in the middle of the discs.

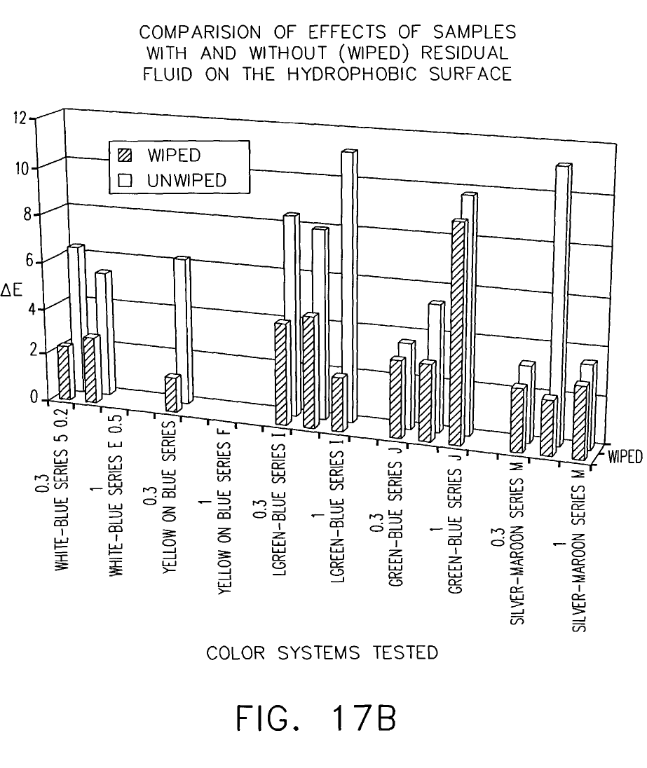

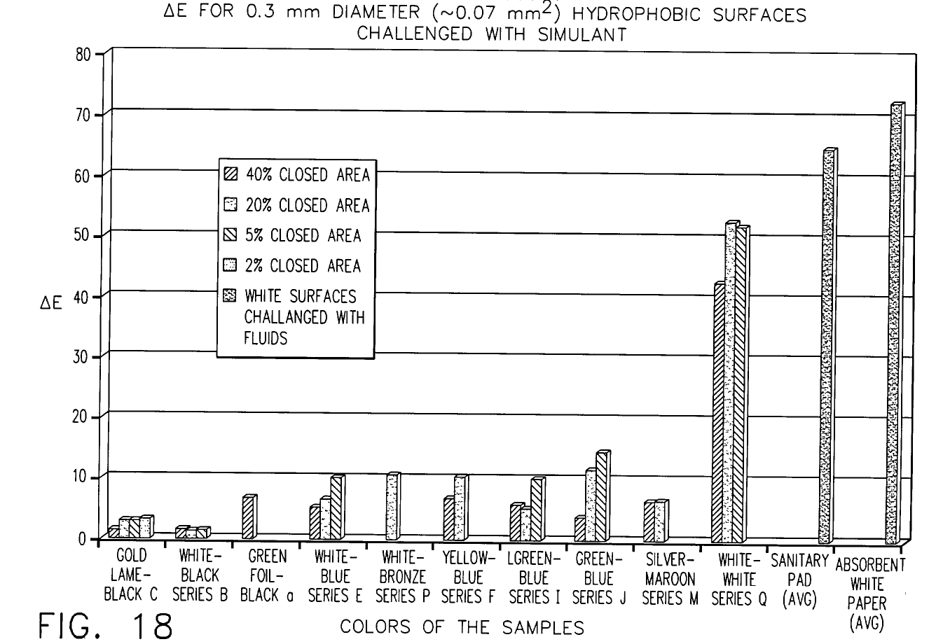

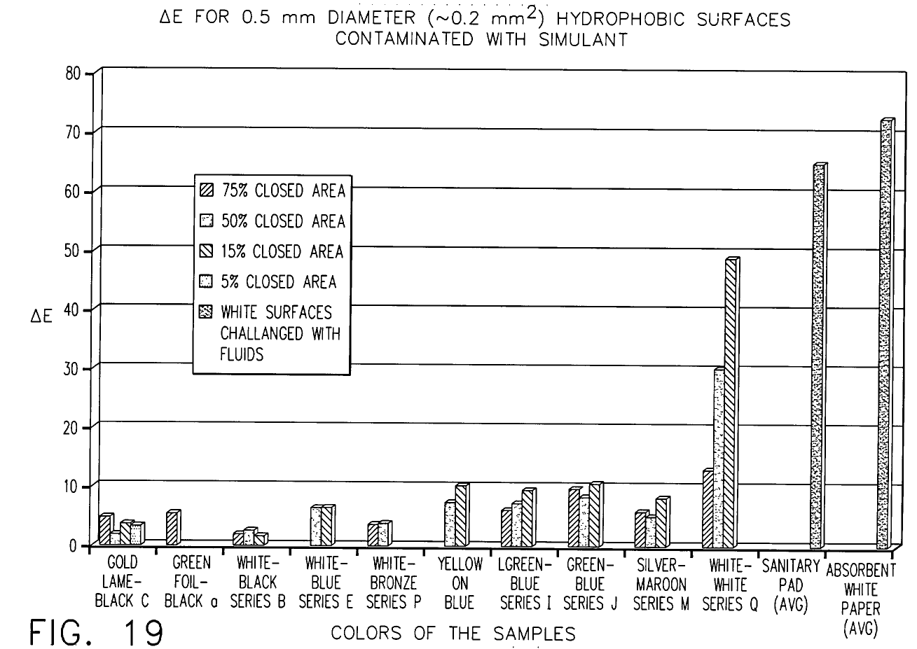

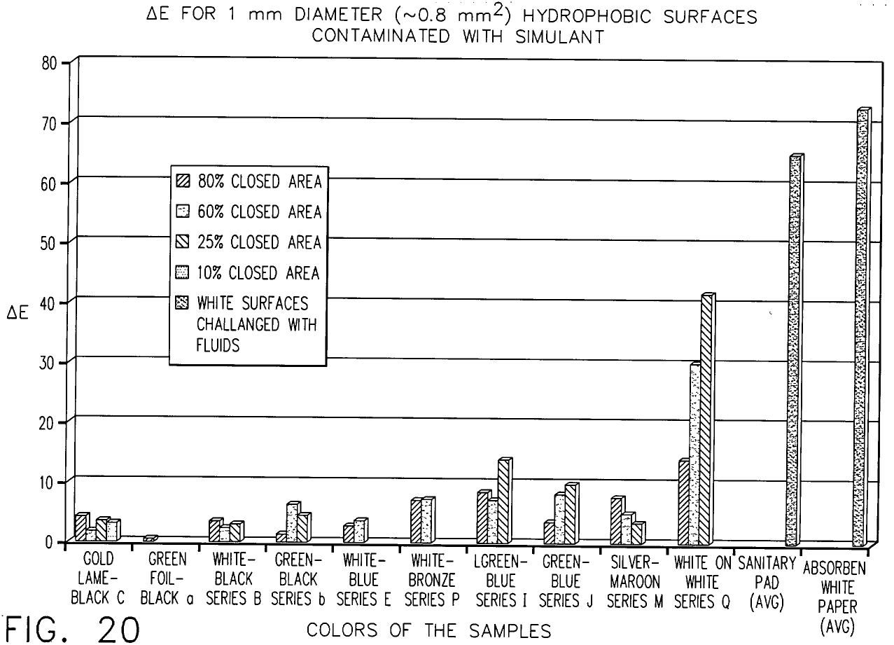

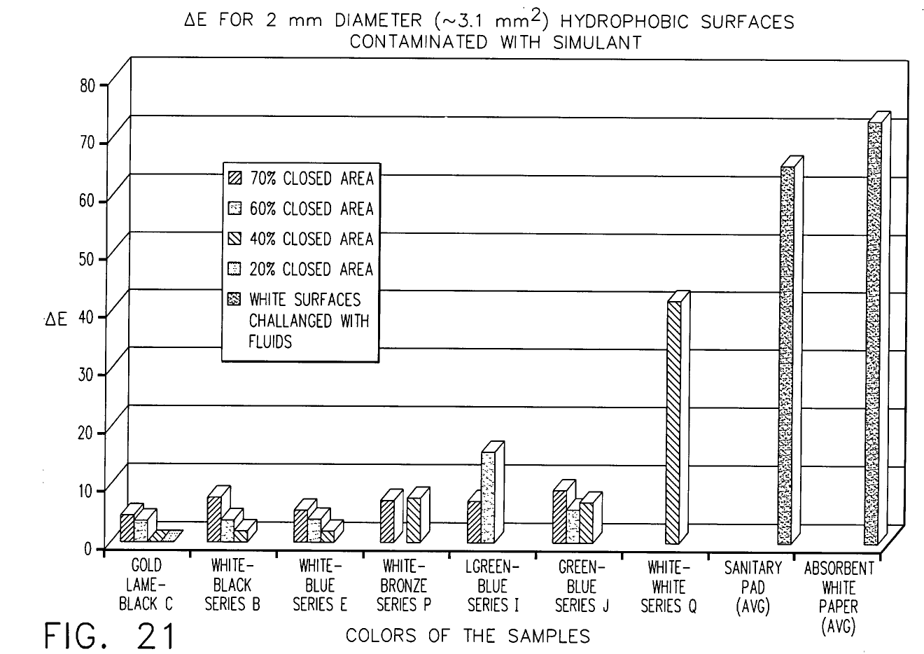

The measured ΔE values in Table 4 represent the change in color reflectivity of a collection of hydrophobic discs and support fabric surfaces within the scanned region. With reference to FIGS. 18-21, it can be seen that acceptable ΔE values were generally achieved for all tested colors, disk sizes and percent area coverages when dark support fabrics were used. FIGS. 18, 19, 20, and 21 summarize and collect the results for tested essentially circular hydrophobic surfaces having diameters of 0.3 mm, 0.5 mm, 1.0 mm, and 2.0 mm respectively. Color masking was even achieved with white hydrophobic surfaces on white support fabric and the color masking was found to be proportional to area coverage due to occluding increasing amounts of stainable, white support fabric.

It can also be seen that for any color and disc size, ΔE and visual perception of stains generally increased as the area coverage decreased regardless of color combinations although this effect was not directly proportional to area coverage. Acceptable ΔE and visual perceptions were observed for disc sizes ranging from 0.3 mm diameter to 2.0 mm diameter, although in certain cases increasing amounts of residual simulant were observed with increasing disc size. However, it was observed that use of very hydrophobic materials such as polymeric fluorocarbons gave rise to minimal or no residual simulant. Thus, a preferred embodiment for larger disc sizes is the use of highly hydrophobic materials such as polymeric fluorocarbons.

Some of the materials were somewhat less color masking than others because of small amounts of residues left on the disc surfaces.

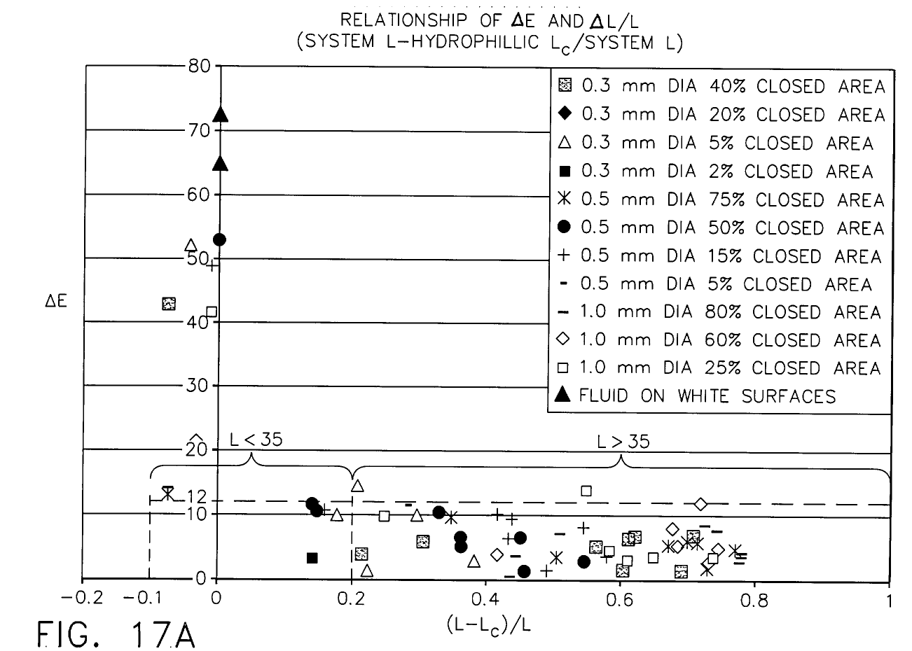

Accordingly, these data are not optimized for color effects because it also has included in it these interferences. If the fluid shedding properties of these materials were improved to shed these residues they would work as if they had been wiped of the residue as shown in FIG. 17B.

Color measurements were also performed using colormasking layers using approximately 0.5 mm diameter green, purple, and red hydrophobic colored foil discs having approximately 0.8 mm separation distances that were deposited on a black fabric of such light weight that one could readily see through one layer. To preclude seeing the red color of AMF behind such layer, three layers of these systems were stacked and measurements were then made. The following ΔE values were obtained for the green, purple, and red colored disc systems: 1.9, 4.1, and 2.6, respectively. These results indicate that multiple colored masking layers can be stacked to attain effective color masking.

With particular attention to the case of the “White on Black Series” above, details of the spacing and disc size are shown below.

Distance of Closest Approach for White Discs on Black
Fabric (Series B) (mm)
Diameter (mm)

| 0.3 mm | 0.5 mm | 1.0 mm | 2.0 mm |
|---|---|---|---|
| 0.2 | 0.1 | 0.1 | 0.2 |
| 0.4 | 0.2 | 0.2 | 0.4 |
| 0.5 | 0.5 | 0.5 | 0.5 |
| 1.0 | 0.9 | 1.0 | 1.0 |
| 2.0 | 1.8 | 2.0 | 2.2 |

As may be seen, effective color masking was achieved where there was great contrast in luminosity between the discs and support fabric, across various disc sizes and spacing.

Interpretation of Luminescence Function. The ΔE versus L_{system}-L_{cloth} system (the luminosity function) plot (FIG. 17A) provides a prediction device to chose hydrophobic surface and support fabric colors. ΔE is small when the relationships as follows, hold: In general, it has been found that when the L value of the absorbent article as a system is greater than 35, and the relationship, (L_{system}−L_{cloth})/L_{system}>0.2, exists between the system and the material of the support fabric, a change in ΔE<˜12 is observed when there is effective color masking, regardless of the color combination, as shown in FIG. 15.

Further, when the L value of the absorbent article as a system is less than 35, and the relationship, (L_{system}−L_{cloth})/L_{system}>−0.1, exists between the system and the material of the support fabric, a change in ΔE<˜12 is observed when there is effective color masking for colors, dark colors and black, as shown in FIG. 15. The negative number takes into account the possibility that a slightly lighter color than the support fabric could be used in the absorbent article system and still a ΔE of<˜10 to 12 can result if the material is dark enough. Typically, when the colors of the support fabric and the hydrophobic surfaces are the same, the relationship, (L_{system}−L_{cloth}/L_{system}) will be equal to 0.

Where a support fabric is not needed to tie together the hydrophobic surfaces, the luminosity of the spaces between the surfaces, Lspaces, may be substituted in the relationships above for the term, Lcloth. In addition, the terms support fabric or cloth are used interchangeably herein to refer to the various materials discussed herein useful in that role in the color masking layer. Similarly, the term, Lcloth, is illustrative, not limiting as to the types of materials used in the color masking layer.

The basic equation ΔE²=ΔL²+Δa²+Δb² shows that ΔE is a function of the luminance and the chromaticity values a and b. The plot of the luminescence function in FIG. 5 shows ΔE as a function of L while ignoring changes in a and b since in this application it can be shown that most of the change in ΔE is due to luminosity rather than chromaticity changes. The differences between the luminosity of the system L and the luminosity of the support fabric are shown to be related to ΔE. The following equation is conceptual but not rigorous: L_{system}=αL′+(α−1)L_{cloth}, where L′ is the luminosity of the hydrophobic discs and α is the fractional area coverage of hydrophobic discs on support fabric (cloth). It can be seen that increasing the luminosity of the hydrophobic discs relative to the luminosity of the support fabric will drive the luminosity function to the right (higher values) while the data show ΔE quickly is reduced to optimally low values, where stains are decreasingly observed. It can also be seen that increasing the luminosity of the support fabric (by using support fabric colors such as white, yellow, green, etc.) relative to the hydrophobic discs will drive this function to the left (to lower or negative values) while for L>35, ΔE is increased into the non-optimal region in which stains are readily visualized. Also, increasing the percentage of hydrophobic disc surface area (α) while maintaining the same L′ and L_{cloth} values can cause the function to move either to the right or to the left. When L′ is larger than L_{cloth}, ,as α is increased, the function will move desirably to the right. Conversely, when L_{cloth} is larger than L′ (as when a bright, light-colored support fabric is used), increasing α will cause the function to move non-desirably to the left while ΔE becomes larger and stains become more visible. All these relationships generally exist regardless of the colors of hydrophobic surfaces and support fabrics.

ΔE is small when the relationships as follows, hold: In general, it has been found that when the L value of the absorbent article as a system is greater than 35, and the relationship, (L_{system}−L_{cloth})/L_{system}>0.2, exists between the system and the material of the support fabric, a change in ΔE<˜12 is observed when there is effective color masking, regardless of the color combination, as shown in FIG. 15.

Further, when the L value of the absorbent article as a system is less than 35, and the relationship, (L_{system}−L_{cloth})/L_{system}>−0.1, exists between the system and the material of the support fabric, a change in ΔE<˜12 is observed when there is effective color masking for colors, dark colors and black, as shown in FIG. 15. The negative number takes into account the possibility that a slightly lighter color than the support fabric could be used in the absorbent article system and still a ΔE of<˜10 to 12 can result if the material is dark enough. Typically, when the colors of the support fabric and the hydrophobic surfaces are the same the relationship, (L_{system}−L_{cloth})/L_{system} will be equal to 0.

Where a support cloth is not needed to tie together the hydrophobic surfaces, the luminosity of the spaces between the surfaces, Lspaces, may be substituted in the relationships above for the term, Lcloth.

The graph of FIG. 17A clearly shows that when the luminosity of the system is greater than the luminosity of the cloth that ΔE is brought quickly into the desirable range with values of less than 12. In fact, the graph indicates that even for a wide range of hydrophobic surfaces and support fabric colors, ΔE falls within this range when the luminosity of the system is about 20 percent greater than the luminosity of the cloth (where L_{system}−L_{cloth}/L_{system} is about 0.2 or greater). The ΔE values of those support fabrics that were black or dark were observed to move more rapidly to lower values as the luminosity function increased. Increasing hydrophobic surface luminosity and decreasing support fabric luminosity so that the luminosity function is greater than approximately 0.2, leads to decreased menstrual fluid perception regardless of the colors of the hydrophobic surfaces and support fabrics. Hydrophobic surface luminosity can be achieved when these surfaces contain pigments having high inherent luminosities, fluorescent pigments, reflective powder or glitter, or when these surfaces have undercoatings composed of reflective or white surfaces. Support fabrics will preferably have relatively low luminosities. Also, increasing the percent area of hydrophobic surfaces will enhance masking of stains when the luminosity of the hydrophobic surfaces is greater than the luminosities of the support fabric.

As shown in Table 5, colored topsheets may further improve color masking abilities of the hydrophobic surface embodiment of the present invention as well. Specifically, and in one example, test results for orange topsheets resulted in increased and observable color masking improvements. The contribution towards color masking by the topsheet is expected to increase as the luminosity of the topsheet is increased to higher values than those used in the current study.

TABLE 5
Topsheets on Color Masking Hydrophobic Surface
Layers after Insult AMF

| Sample Designation | | | |
|---|---|---|---|
| (Colored Topsheet on | Top- | | |
| Hydrophobic Disc System/Fabric | sheet | | |
| Color | L | ΔE | Observations |
| Orange Tredegar 22Hex on | 59 | 2.89 | Not observable |
| Yellow Chromatec/Blue | | | |
| No Topsheet on Yellow | — | 12.93 | Low contrast stain, |
| Chromatec/Blue | | | observable |
| Orange Tredegar HexPent on | 59 | 8.84 | Barely observable, |
| Yellow Chromatec/Bronze | | | not observable at an |
| | | | angle |
| No Topsheet on Yellow | — | 9.27 | Barely observable |
| Chromatec/Bronze | | | |

In addition, printed patterns on topsheets, such as patterns or camouflage patterns can further serve to enhance the performance of the color masking layer. Data in Table 6 below describes the effect of the addition of AMF to three commercially available systems having multiple color patterns. One of the samples is a camouflaged fabric, composed of irregular splotches of green, brown, black and tan, and the other two samples consisted of a hydrophobic colored disks bearing various color patterns. One of these samples (Tinkerbell) has multiple colors, where the same color covers adjacent disks, while the other disk system has a hologram pattern superimposed over various interference patterns. As may be seen in the Table below, the observer can barely observe or not observe a stain after application of AMF. The common feature of each material is that they have many adjacent colors which helps to confuse the eye before and after application of AMF. These observations are supported by multiple reflectance measurements taken over many locations in each sample resulting in relatively small ΔE values that were smaller than their standard deviations. Thus, the stains are either barely or not detected visually, as the eye is also presented with multiple local visual effects.

TABLE 6
Use of Patterns with Color Masking Techniques

| | Before Application | | | After Application of Artificial | | | | |
|---|---|---|---|---|---|---|---|---|
| | of Artificial | | | Menstrual Fluid | | | | |
| | Menstrual Fluid | | | | | No. | | |
| | | Std. | No. of | | Std. | of | | |
| SAMPLE | E | Dev. | Tests | E | Dev. | Tests | ΔE | Observation |
| Com'l. Camouflaged | 38.5 | 8.5 | 19 | 30.8 | 5.4 | 12 | −7.7 | Barely |
| Fabric | | | | | | | | Observable |
| Tinkerbell - multicolored | 63.5 | 0.5 | 4 | 63.6 | 0.6 | 3 | 0.1 | Not |
| hydrophobic surface on | | | | | | | | Observable |
| black fabric | | | | | | | | |
| Foxy Spandex - variably | 45.4 | 12 | 3 | 42.9 | 14 | 3 | 2.5 | Not |
| colored design on black | | | | | | | | Observable |
| fabric | | | | | | | | |

When topsheets were placed above a variety of color masking layers consisting of regularly spaced colored hydrophobic disks, a variety of Moire' patterns were generated which served to positively modify the bright colors that help make the color masking effect work to gives a more pleasing visual appearance before and after use.

It is further understood in accordance with the present invention that the color masking layer may be configured as two or more color masking layers whether such layers are made of a more gauze like support fabric with widely spaced hydrophobic or liquid impermeable areas, or more closely spaced areas. The presence of two or more layers permits more open materials to be used advantageously, and can also be applied in ways that prevent remaining portions of the adsorbent article from being visible to the user.

In an alternative embodiment of the present invention, the color masking layer may comprise a first masking material which includes a plurality of opaque areas, preferably covering 50% or more of the first masking material area, disposed on a transparent or translucent apertured support; and a second masking material comprising a color masking fabric layer which underlies the apertured support. In this embodiment, it is preferred that the L value of the second masking material as viewed through the transparent or translucent apertured support be less than the L value of the first color masking material. The second masking material may further include hydrophobic areas or fluid impermeable areas separated by fluid permeable spaces in accordance with the present invention. In a still further embodiment of the present invention a color masking layer may be provided in a sleeve configuration, where the color masking layer is provided on at least one surface. The sleeve may be either disposable or washable, and the remaining portion of an absorbent article, such as an absorbent pad, may be placed inside the sleeve. The remaining portion of the absorbent article may, in turn, be either reusable or disposable. The sleeve may be constructed for loading from the narrow end, or by a longitudinal slit, in either case preferably made in a manner which includes a flap to overlap the opening to assist in retaining the inserted element. In this embodiment, a disposable or reusable top sheet may also be provided.

A further aspect of the present invention provides for removability of the color masking layer, or removability (when used in the device) of a top sheet. This facilitates various product forms where the top sheet or masking layer may advantageously be disposed of, retained or reused, and separable from other portions of the absorbent article. These layers may optionally be removably attached, such as by adhesives, hook and loop fasteners, press fit attachments, and the like. The present invention is particularly useful in its application to personal hygiene and health products, such as feminine sanitary pads, tampons, pantyliners, wound dressings and bandages.

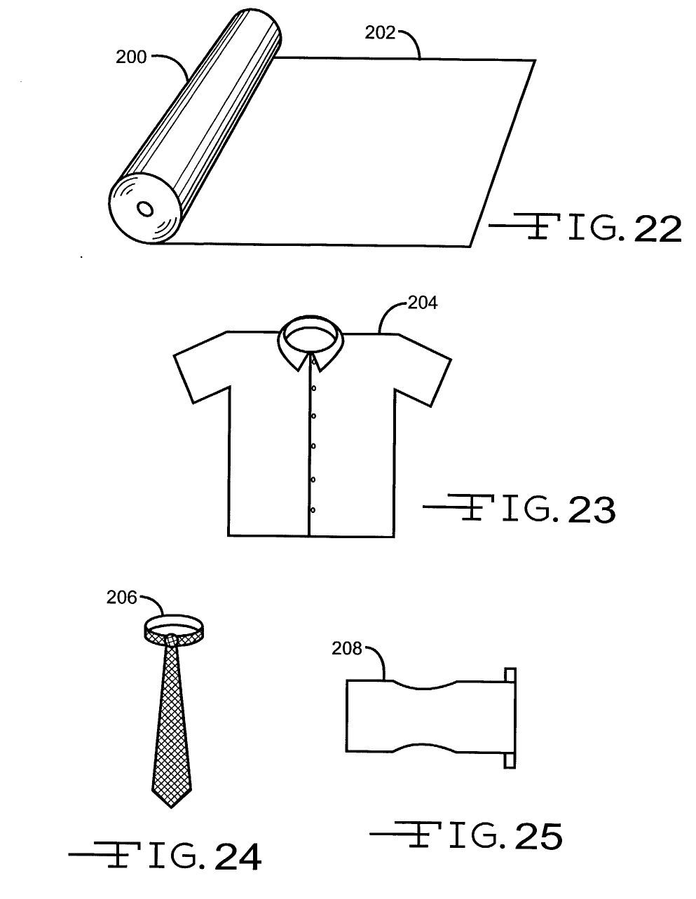

In other embodiments of the invention, the color masking features of the invention are applied to fabrics for a wide variety of uses to provide superior stain masking qualities for the fabrics. A roll 200 of fabric 202 treated or made according to the invention is shown in FIG. 22. As an example of one of the many uses of fabrics treated or made according to the invention, the fabric can be used for wearing apparel, which includes but is not limited to outer clothing, underwear, aprons, neckties, hats, shoes, sports uniforms, outdoor apparel, outdoor equipment (tents, backpacks, awnings and outdoor furniture), industrial apparel including uniforms, apparel for the very young (bibs) and apparel for the elderly. A shirt 204 treated or made according to the invention is shown in FIG. 23, and a necktie 206 treated or made according to the invention is illustrated in FIG. 24. Fabrics for any of these uses that are treated or made according to the invention for improved color masking will have enhanced value because of the reduced tendency for the fabric to show stains or spots from such occurrences as food and condiment spills, juice and wine spills, blood stains, ink spots, and dirt or mud spots.

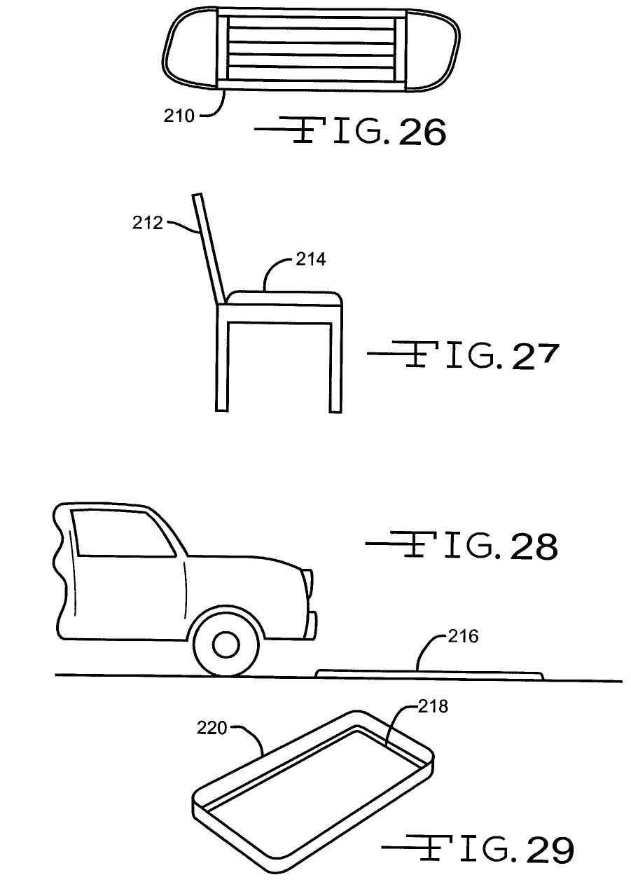

Another area where the invention can be useful in the area of therapeutic products, which is defined as medical apparel including hospital and surgical gowns, surgical drapes and curtains. Therapeutic products also includes surgical and medical pads for use on animals. Therapeutic products further includes diapers (both adult and children), wipes, and sweat pads, as well as sanitary pads and diapers for animals, such as household pets. FIG. 25 shows a diaper 208 treated or made according to the invention. A surgical face mask 210 is illustrated in FIG. 26. The for purposes of the invention, the term “therapeutic products” includes any of these uses disclosed in this paragraph, including diapers configured for use on humans, but not including personal hygiene absorbent products such as sanitary napkins, pads or bandages configured for use on humans.

Fabrics treated or made according to the invention can also be used for decorative product applications. One such decorative product application of the inventive fabric is in upholstery, such as for use on furniture and cushions, and in automotive and airplane interiors. FIG. 27 illustrates a chair 210 upholstered with a fabric 214 according to the invention. One particular use according to the invention is a stain masking absorbent pad for use as a liner in a cupholder in an automobile or other vehicle. Other decorative product applications include interior decorations wall coverings, such as in drapes, curtains and decorative fabrics, and floor coverings, such as carpets, rugs, door mats and floor mats. Additional decorative applications of the invention include towels, linens, table cloths, napkins, handkerchiefs, and bedding materials, such as sheets, bed pads and blankets.

Fabrics or other fibrous or non-fibrous material treated or made according to the invention can also be used as floor liners for collecting and hiding grease spots or other unsightly stains. This could be used in an automobile garage to hide or mask grease spots from an automobile. The invention could also be used for hiding grease spots in a service garage, such as in automobile maintenance shops. Industrial facilities could also be provided with liners or pads treated or made according to the invention to hide stains from grease or any other industrial material. A grease absorbing floor liner or pad 216 is shown in FIG. 28. For an oil-based or oil soluble spilled material, the preferred product of the invention has an interior liquid-absorbing portion of hydrophobic or oleophilic material and a wear surface portion having particles of hydrophilic or oleophobic material. Preferably, the reflective particles cover an amount of the surface of the within the range of from about 50 to about 90 percent of the surface area of the product, and where the particles are substantially more reflective than the hydrophilic material.

There is an application of material treated with or made according to the invention in the food industry. Packaging material provided with the stain masking properties of the invention would be beneficial, particularly for meat and fish, but also for other food products. A food packaging mat 218 inside a food tray 220 is shown in FIG. 29. Packaging material according to the present invention may further be configured to adsorb and prevent intrusion of fluids into a shipped article during transport.

While the invention has been disclosed as being primarily a disposable product for some of the product applications disclosed above, e.g. sanitary napkins, diapers and food packaging products, it is to be understood that the surface or color masking layer can be made in a manner to be replaced. This will allow the product to be refreshed for further use. For example, a floor mat for masking oil dripping from an automobile could be provided with a detachable color masking layer. The detachable color masking layer can be replaced with a new color masking layer, or alternatively the layer can be washed and repositioned on the floor mat. Still further, the color masking layer can be provided as a sleeve to decoratively enclose either a washable or disposable

What is claimed is:

1. An adsorbent fabric comprising a color masking layer including fluid impermeable areas disposed on a fluid permeable support fabric in spaced relationship.

2. Wearing apparel made with the fabric of claim 1.

3. A therapeutic product comprising a color masking layer including fluid impermeable areas disposed on a fluid permeable support fabric in spaced relationship.

4. A decorative product comprising a color masking layer including fluid impermeable areas disposed on a fluid permeable support fabric in spaced relationship.

5. An upholstery fabric according to claim 4.

6. Interior decorations according to claim 4.

7. A floor liner comprising a color masking layer including fluid impermeable areas disposed on a fluid permeable support fabric in spaced relationship, and further including an interior liquid-absorbing portion of hydrophobic or oleophilic material, and wherein the fluid impermeable areas comprise particles of hydrophilic or oleophobic material

8. Packaging material comprising a color masking layer including fluid impermeable areas disposed on a fluid permeable support fabric in spaced relationship.

9. An adsorbent fabric having a color masking layer comprising a plurality of areas comprised of fluid impermeable material separated by fluid permeable spaces, wherein the fluid impermeable material has a luminosity greater than that of the fluid permeable spaces.

10. Wearing apparel made with the fabric of claim 9.

11. A therapeutic product having a color masking layer comprising a plurality of areas comprised of fluid impermeable material separated by fluid permeable spaces, wherein the fluid impermeable material has a luminosity greater than that of the fluid permeable spaces.

12. A decorative product having a color masking layer comprising a plurality of areas comprised of fluid impermeable material separated by fluid permeable spaces, wherein the fluid impermeable material has a luminosity greater than that of the fluid permeable spaces.

13. An upholstery fabric according to claim 12.

14. Interior decorations according to claim 12.

15. A floor liner having a color masking layer comprising a plurality of areas comprised of fluid impermeable material separated by fluid permeable spaces, wherein the fluid impermeable material has a luminosity greater than that of the fluid permeable spaces, and further including an interior liquid-absorbing portion of hydrophobic or oleophilic material, and wherein the fluid impermeable areas comprise particles of hydrophilic or oleophobic material

16. Packaging material having a color masking layer comprising a plurality of areas comprised of fluid impermeable material separated by fluid permeable spaces, wherein the fluid impermeable material has a luminosity greater than that of the fluid permeable spaces.

17. An adsorbent fabric comprising an apertured topsheet having an L value greater than 60 and a color masking fabric layer underlying the topsheet.

18. Wearing apparel made with the fabric of claim 17.

19. A therapeutic product comprising an apertured topsheet having an L value greater than 60 and a color masking fabric layer underlying the topsheet.

20. A decorative product comprising an apertured topsheet having an L value greater than 60 and a color masking fabric layer underlying the topsheet.

21. An upholstery fabric according to claim 20.

22. Interior decorations according to claim 20.

23. A floor liner comprising an apertured topsheet having an L value greater than 60 and a color masking fabric layer underlying the topsheet, and further including an interior liquid-absorbing portion of hydrophobic or oleophilic material, and wherein the topsheet includes hydrophilic or oleophobic material

24. Packaging material comprising an apertured topsheet having an L value greater than 60 and a color masking fabric layer underlying the topsheet.

25. An adsorbent fabric comprising a masking layer including (a) a first masking material comprising a plurality of opaque areas disposed on a substantially transparent, apertured support, and (b) a second masking material comprising a color masking fabric layer underlying the support layer.

26. Wearing apparel made with the fabric of claim 25.

27. A therapeutic product comprising a masking layer including (a) a first masking material comprising a plurality of opaque areas disposed on a substantially transparent, apertured support, and (b) a second masking material comprising a color masking fabric layer underlying the support layer.

28. A decorative product comprising a masking layer including (a) a first masking material comprising a plurality of opaque areas disposed on a substantially transparent, apertured support, and (b) a second masking material comprising a color masking fabric layer underlying the support layer.

29. An upholstery fabric according to claim 28.

30. Interior decorations according to claim 28.

31. A floor liner comprising a masking layer including (a) a first masking material comprising a plurality of opaque areas disposed on a substantially transparent, apertured support, and (b) a second masking material comprising a color masking fabric layer underlying the support layer, and further including an interior liquid-absorbing portion of hydrophobic or oleophilic material, and wherein the topsheet includes hydrophilic or oleophobic material

32. Packaging material comprising a masking layer including (a) a first masking material comprising a plurality of opaque areas disposed on a substantially transparent, apertured support, and (b) a second masking material comprising a color masking fabric layer underlying the support layer.
